(19) United States
(12) Patent Application Publication
(10) Pub. No.: US 2021/0347794 A1
(43) Pub. Date: Nov. 11, 2021

(54) Phosphate Derivatives of Indole Compounds and Their Use

(71) Applicant: ARIAGEN, INC., Menlo Park, CA (US)

(72) Inventors: Graham Johnson, Sanbornton, NH (US); David Douglas Manning, Orchard Park, NY (US)

(73) Assignee: ARIAGEN, INC., Menlo Park, CA (US)

(21) Appl. No.: 17/278,579

(22) PCT Filed: Sep. 23, 2019
(86) PCT No.: PCT/US2019/052494

§ 371 Date: Mar. 22, 2021

Related U.S. Application Data
(60) Provisional application No. 62/734,989, filed on Sep. 21, 2018.

(51) Int. Cl. C07F 9/24; C07F 9/141; A61P 35/00

(52) U.S. Cl. CPC C07F 9/247; A61P 35/00; C07F 9/1415

(57) ABSTRACT

The present disclosure relates to phosphate derivatives of indole compounds and pharmaceutical compositions thereof, and their use in stimulating the immune system of patients such as cancer patients.

### CROSS-REFERENCE TO RELATED APPLICATION

This application claims priority from U.S. Application 62/734,989, filed Sep. 21, 2018, the content of which is incorporated herein by reference in its entirety.

### FIELD OF THE INVENTION

The present disclosure relates to phosphate derivatives of indole compounds and their use in treating patients in need thereof, such as patients with cancer or in need of immune stimulation.

### BACKGROUND OF THE INVENTION

The aryl hydrocarbon (Ah) receptor (AhR) is a ligand-inducible transcription factor and a member of the basic helix-loop-helix/Per-Arnt-Sim (bHLH/PAS) superfamily. Upon binding to its ligand, AhR mediates a series of biological processes, including cell division, apoptosis, cell differentiation, adipose differentiation, hypothalamus actions, angiogenesis, immune system modulation, teratogenicity, tumorigenicity, tumor progression, chloracne, wasting, actions of hormonal systems (e.g., estrogen and androgen), and expression of genes of the P450 family (Poland et al., Annu. Rev. Pharmacol. Toxicol. 22:517-554 (1982); Poellinger et al., Food Addit Contam. 17(4):261-6 (2000); Bock et al., Biochem. Pharmacol. 69(10):1403-1408 (2005); Stevens et al., Immunology 127(3):299-311 (2009); Puga et al., Biochem Pharmacol. 69(2):199-207 (2005); Safe et al., Int J Oncol. 20(6):1123-8 (2002); Dietrich et al., Carcinogenesis 31(8):1319-1328 (2010); U.S. Pat. No. 7,419,992). The liganded receptor participates in biological processes through translocation from cytoplasm into the nucleus, heterodimerization with another factor named Ah receptor nuclear translocator, and binding of the heterodimer to the Ah response element of AhR-regulated genes, resulting in enhancement or inhibition of transcription of those genes.

The AhR is able to bind, with different affinities, to several groups of exogenous chemicals, or artificial ligands, including polycyclic aromatic hydrocarbons, e.g., 3-methylchoranthrene (3-MC), and halogenated aromatic hydrocarbons, e.g., 2,3,7,8-tetrachlorodibenzo-p-dioxin (TCDD). Studies with those AhR artificial ligands have helped in advancing the understanding of the AhR system. An endogenous or physiological ligand for the AhR has been identified as 2-(1′H-indole-3′-carbonyl)-thiazole-4-carboxylic acid methyl ester (ITE), with the following structure:

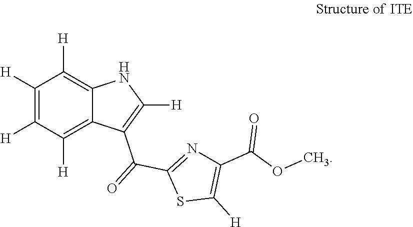 [SMILES: COC(=O)c1csc(C(=O)c2c[nH]c3ccccc23)n1]

See, e.g., Song et al., PNAS USA 99(23):14694-9 (2002); and U.S. Pat. No. 6,916,834.

### SUMMARY OF THE INVENTION

The present disclosure provides phosphate derivatives of indole compounds that can be useful in modulating an activity of the human aryl hydrocarbon receptor (AhR), pharmaceutical compositions comprising one or more of these compounds, use of these compounds and compositions in treating diseases and conditions in patients who can benefit from modulation of AhR activities. The phosphate derivative of an indole compound can include a phosphate moiety, which can be a phosphate salt. The phosphate moiety can include an alkoxy group. The phosphate salt can have one or more counter ions, which can be an alkali metal ion, an alkaline earth metal ion, or an organic amine cation.

The compounds can be an indolo-phosphoramidate analog (IPA). The indolo-phosphoramidate analog can have a nitrogen-phosphorous (N—P) bond. In certain embodiments, the indolo-phosphoramidate analog can include a labile linker between the indole nitrogen and the phosphate phosphorus. For example, an indolo-phosphoramidate analog can include an alkyloxy group as a labile linker between the indole nitrogen and the phosphate phosphorous, such as a hydroxymethylphosphate (HMP) derivative. The linker can form a phosphate. Alternatively, the linker can be non-labile, such as a phosphonate. The labile linker can be of the formula —(CR₂R₃—O)ₓ—, where x is 0, 1, 2, 3, 4, 5, or 6 and each of R₂ and R₃ can be, independently, H or C₁-C₆ alkyl. The carbon of the CR₂R₃—O— group can be bonded to the indole nitrogen. In certain embodiments, x is 0 or 1. In certain embodiments, each of R₂ and R₃ can be, independently, H.

In one aspect, a compound can have Formula I:

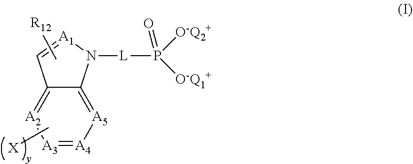

R₁₂ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Each of A₁, A₂, A₃, A₄, and A₅, independently, can be CR₂ or N.

L can be —(CR₂R₃—O)ₙ— or a bond.

R₂ can be H or C₁-C₆ alkyl, R₃ can be H or C₁-C₆ alkyl, or, together, R₂ and R₃ can form a C₃-C₈ cycloalkyl.

n can be 0, 1, 2, 3, 4, 5, or 6.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Q₁⁺ and Q₂⁺ can be each, independently, a monocation, or together can be a dication or one or both of Q₁⁺ or Q₂⁺ can be H, C₁-C₆ alkyl, benzyl, allyl, or —(CR₂R₃—O)—R₂₃, and R₂₃ can be H or C₁-C₆ alkyl. The alkyl can be a substituted alkyl, for example an alkoxy alkyl, amino alkyl, alkyl ester, alkyl carbamate, or alkyl carbonate.

For clarity, and as described herein, when one or both of Q₁⁺ or Q₂⁺ is H, C₁-C₆ alkyl, benzyl, allyl, or —(CR₂R₃—O)—R₂₃, and R₂₃ can be H or C₁-C₆ alkyl, —O⁻Q₁⁺ or —O⁻Q₂⁺ depicts a covalent bond. In certain circumstances, a —P—OH group may not be ionized.

In one aspect, a compound can have a structure of Formula II:

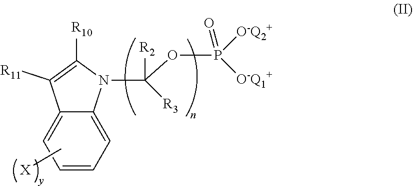

R₁₀ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

R₁₁ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

One of R₁₀ and R₁₁ is H or C₁-C₆ alkyl.

R₂ can be H or C₁-C₆ alkyl, R₃ can be H or C₁-C₆ alkyl, or, together, R₂ and R₃ can form a C₃-C₈ cycloalkyl.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Q₁⁺ and Q₂⁺ can be each, independently, a monocation, or together are a dication or one or both of Q₁⁺ or Q₂⁺ can be H, C₁-C₆ alkyl, benzyl, allyl, or —(CR₂R₃—O)—R₂₃, and R₂₃ can be H or C₁-C₆ alkyl, and the other of Q₁⁺ or Q₂⁺ can be a monocation. The alkyl can be a substituted alkyl, for example an alkoxy alkyl, amino alkyl, alkyl ester, alkyl carbamate, or alkyl carbonate.

n can be 0, 1, 2, 3, 4, 5, or 6, preferably, 0 or 1.

In certain circumstances, the compound can be of Formula III:

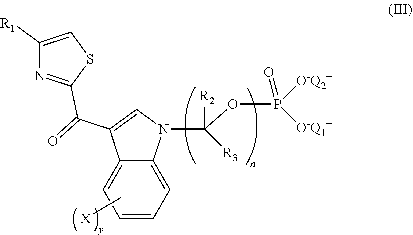

wherein:

R₁ can be —C(═O)—R₄, cyano, an oxadiazole, or a thiadiazole, wherein the oxadiazole, or the thiadiazole can be optionally substituted by amino, alkyl amino, amino alkyl, alkoxy, alkyl, or haloalkyl.

R₂ and R₃ can be each, independently, hydrogen, or C₁-C₆ alkyl.

R₄ can be selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyl oxy, halothiocarbonyloxy, thiocarbonylthio, or halothiocarbonylthio.

Q₁⁺ and Q₂⁺ can be each, independently, a monocation, or together are a dication or one or both of Q₁⁺ or Q₂⁺ can be H, C₁-C₆ alkyl, benzyl, allyl, or —(CR₂R₃—O)—R₂₃, and R₂₃ can be H or C₁-C₆ alkyl, and the other of Q₁⁺ or Q₂⁺ can be a monocation. The alkyl can be a substituted alkyl, for example an alkoxy alkyl, amino alkyl, alkyl ester, alkyl carbamate, or alkyl carbonate.

n can be 0, 1, 2, 3, 4, 5, or 6, preferably, 0 or 1.

In certain circumstances, the compound can be of Formula IV:

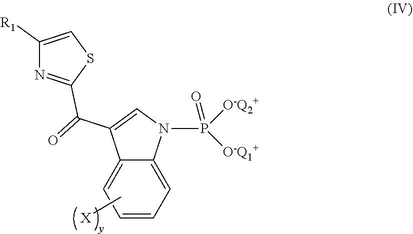

wherein:

R₁ can be —C(═O)—R₄, cyano, an oxadiazole, or a thiadiazole.

R₄ can be selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Q₁⁺ and Q₂⁺ can be each, independently, a monocation, or together are a dication or one or both of Q₁⁺ or Q₂⁺ can be H, C₁-C₆ alkyl, benzyl, allyl, or —(CR₂R₃—O)—R₂₃, and R₂₃ can be H or C₁-C₆ alkyl, and the other of Q₁⁺ or Q₂⁺ can be a monocation. The alkyl can be a substituted alkyl, for example an alkoxy alkyl, amino alkyl, alkyl ester, alkyl carbamate, or alkyl carbonate.

In certain circumstances, the compound can be of Formula V:

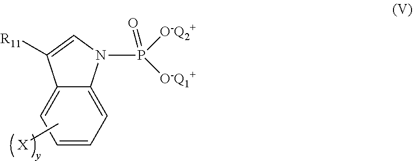

wherein:

R₁₀ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

R₁₁ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

One of R₁₀ and R₁₁ is H or C₁-C₆ alkyl.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Q₁⁺ and Q₂⁺ can be each, independently, a monocation, or together are a dication or one or both of Q₁⁺ or Q₂⁺ can be H, C₁-C₆ alkyl, benzyl, allyl, or —(CR₂R₃—O)—R₂₃, and R₂₃ can be H or C₁-C₆ alkyl, and the other of Q₁⁺ or Q₂⁺ can be a monocation. The alkyl can be a substituted alkyl, for example an alkoxy alkyl, amino alkyl, alkyl ester, alkyl carbamate, or alkyl carbonate.

In certain circumstances, Q₁⁺ and Q₂⁺ can be each, independently, H or an alkali metal.

In certain circumstances, Q₁⁺ and Q₂⁺ can be each, independently, selected from the group consisting of lithium, sodium, potassium, ammonium, and alkyl ammonium.

In certain circumstances, Q₁⁺ and Q₂⁺ together can be an alkaline earth metal salt.

In certain circumstances, Q₁⁺ and Q₂⁺ can be each independently selected from the group consisting of zinc, calcium, and magnesium.

In certain circumstances, Q₁⁺ and Q₂⁺ can be each independently lithium, sodium, or potassium, y can be 0, 1, or 2, and X can be F, Cl, or Br.

In certain circumstances, the compound can be selected from the group consisting of:

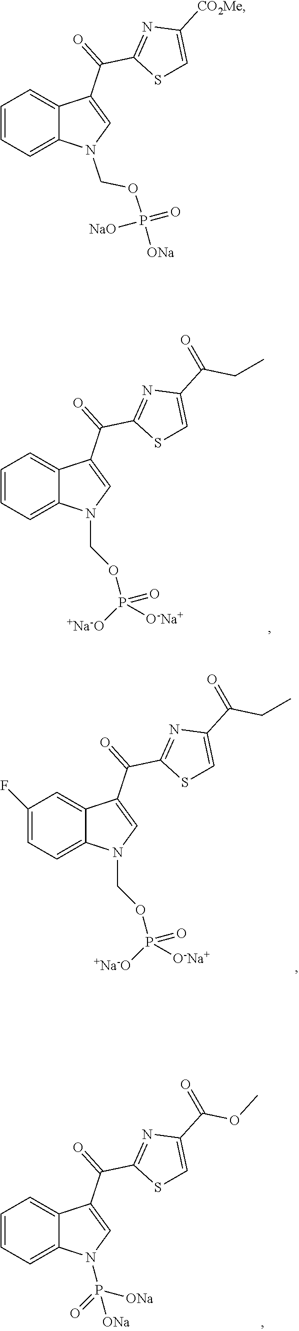

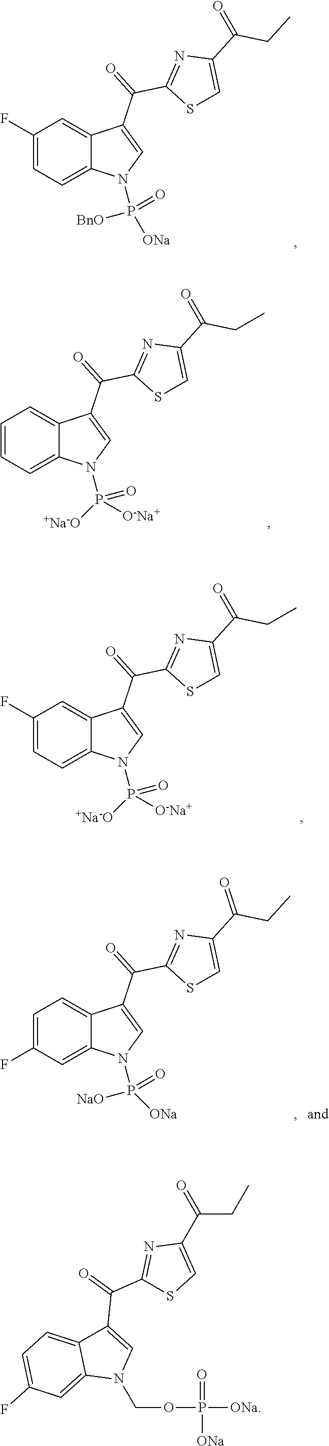

In certain circumstances, the compound can be selected from the group consisting of:

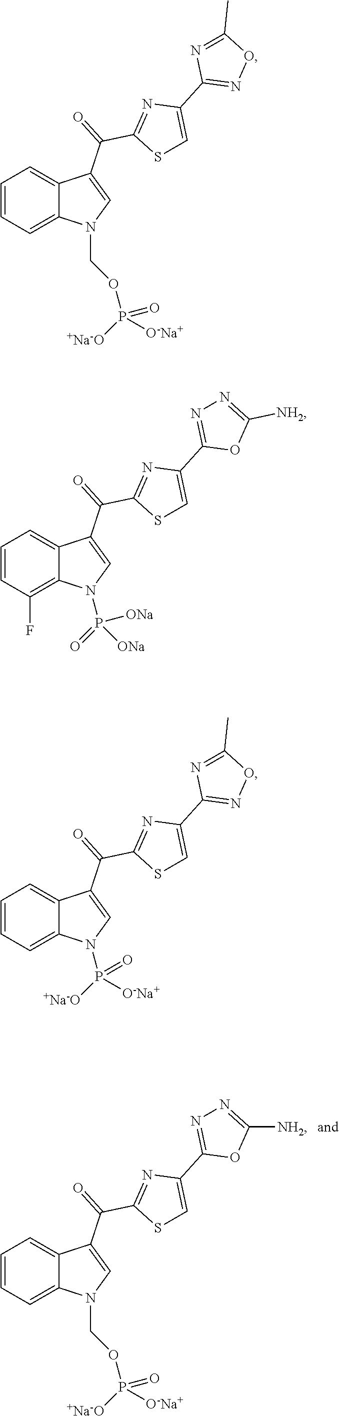

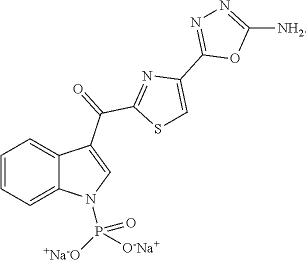

In certain circumstances, a compound can be of Formula VI:

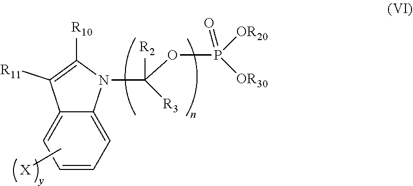

R₁₀ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

R₁₁ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

One of R₁₀ and R₁₁ can be H or C₁-C₆ alkyl.

R₂ can be H or C₁-C₆ alkyl, R₃ can be H or C₁-C₆ alkyl, or, together, R₂ and R₃ form a C₃-C₈ cycloalkyl.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

R₂₀ and R₃₀ each, independently, can be H, C₁-C₆ alkyl, allyl, or benzyl, or one of R₂₀ or R₃₀ is H, C₁-C₆ alkyl, allyl, or benzyl and the other of R₂₀ or R₃₀ is a cation.

n can be 0, 1, 2, 3, 4, 5, or 6. Preferably, n can be 0 or 1.

In certain circumstances, R₁ can be —C(═O)—R₄, and R₄ is C₁-C₆ alkyl or C₁-C₆ alkoxy.

In certain circumstances, R₁ can be an oxadiazole or a thiadiazole.

In certain circumstances, n can be 0, 1, or 2.

In certain circumstances, R₁ can be an unsubstituted or substituted oxadiazole.

In certain circumstances, the oxadiazole, or the thiadiazole can be optionally substituted by amino, alkyl amino, amino alkyl, alkoxy, alkyl, or haloalkyl. For example, the oxadiazole can be substituted with an amino or amino methyl group.

In certain circumstances, the indole is a fluorinated indole. For example, the fluorinated indole can be a 7-fluoro-indole.

In certain circumstances, n can be 0.

In certain circumstances, Q₁⁺ and Q₂⁺ each can be lithium, sodium, or potassium.

In certain circumstances, a method of enhancing the immune system in a patient in need thereof can include administering to the patient a therapeutically effective amount of the compound described herein.

In certain circumstances, a method of treating cancer in a patient in need thereof can include administering to the patient a therapeutically effective amount of the compound of described herein.

The present disclosure also provides a pharmaceutical composition comprising a compound described herein and a pharmaceutically acceptable carrier.

The present disclosure provides a method of stimulating the immune system in a patient in need thereof, comprising administering to the patient a therapeutically effective amount of a compound described herein. In some embodiments, the patient has an increased count of cells selected from the group consisting of white blood cells, macrophages, neutrophils, lymphocytes (e.g., B lymphocytes and/or T lymphocytes), natural killer (NK) cells, dendritic cells, and platelets, or increased levels of cytokines (indicative of a stimulated immune system) after the administering step.

The present disclosure also provides a method of treating cancer in a patient, comprising administering to the patient a therapeutically effective amount of a compound described herein. In some embodiments, the cancer is a hematological malignancy (e.g., a lymphoma, leukemia, or myeloma), or a solid tumor. In some embodiments, the cancer is selected from the group consisting of diffuse large B-cell lymphoma, marginal zone lymphoma, chronic lymphocytic leukemia (CLL), small lymphocytic lymphoma, prolymphocytic leukemia, acute lymphocytic leukemia, Waldenstrom's Macroglobulinemia (WM), follicular lymphoma, mantle cell lymphoma (MCL), Hodgkin lymphoma, non-Hodgkin lymphoma, multiple myeloma, prostate cancer, ovarian cancer, fallopian tube cancer, cervical cancer, breast cancer, lung cancer (e.g., non-small cell lung cancer), skin cancer (e.g., melanoma), colorectal cancer, stomach cancer, pancreatic cancer, liver cancer, kidney cancer, bladder cancer, soft tissue cancer, glioma, and head and neck cancer. In some embodiments, the method further comprises administering to the patient another cancer therapeutic agent, e.g., an immune checkpoint inhibitor (e.g., a PD-1, PD-L1, and/or PD-L2 inhibitor). In some embodiments, the method further comprises administering one or more maintenance doses of the compound while the patient is in remission.

Also provided here is a compound or pharmaceutical composition described herein for use in stimulating the immune system or treating cancer in a patient in need thereof in a treatment method described herein.

The present disclosure further provides the use of a compound described herein for the manufacture of a medicament for stimulating the immune system or treating cancer in a patient in need thereof in a treatment method described herein.

### BRIEF DESCRIPTION OF THE DRAWINGS

FIG. 1 depicts a scheme depicting a synthetic approach to indolo-phosphoramidate analogs.

FIG. 2 depicts a scheme showing an alternative synthetic approach to indolo-alkyloxyphosphate analogs that are examples of analogs of indolophophoramidates.

FIG. 3 depicts a scheme showing an alternative synthetic approach to indolo-alkyloxyphosphate analogs.

FIG. 4 depicts a synthetic approach to an indolo-phosphoramidate analog.

FIG. 5 depicts a synthetic approach to an indolo-alkyloxyphosphate analog.

FIG. 6 depicts a synthetic approach to an indolo-phosphoramidate analog.

FIG. 7 depicts a synthetic approach to an indolo-phosphoramidate analog.

FIG. 8 depicts a synthetic approach to an indolo-phosphoramidate analog.

FIG. 9 depicts a synthetic approach to an indolo-phosphoramidate analog.

FIG. 10 is a graph comparing the tumor inhibitory activities of ARI-158 and ARI-160 in the EMT-6 syngeneic mouse tumor model.

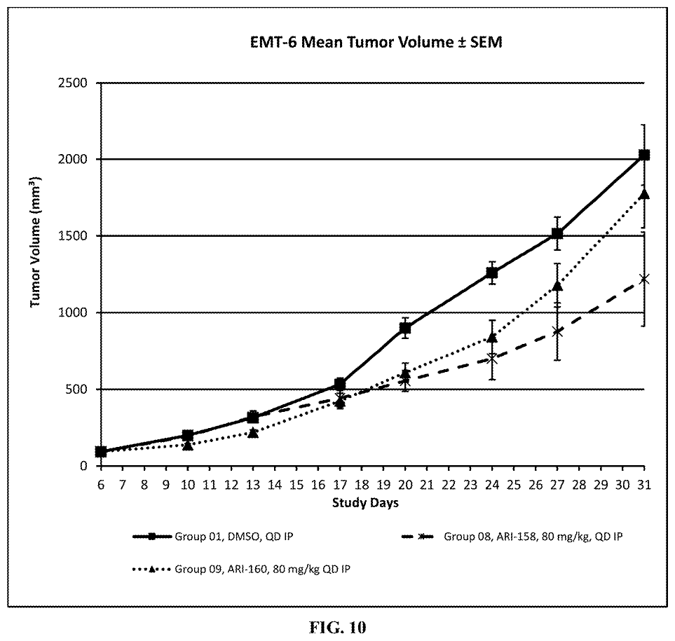

### DETAILED DESCRIPTION OF THE INVENTION

All technical and scientific terms used herein are the same as those commonly used by those ordinary skilled in the art to which the present invention pertains unless defined specifically otherwise.

The term “prodrug” refers to a compound that can undergo biotransformation, e.g., in the body of a human patient, prior to exhibiting its pharmacological action. See Prodrugs; Challenges and Rewards, Volumes 1 and 2, V. Stella, R. T. Borchardt, M. J. Hageman, R. Oziyai, H. Maag, and J. W. Tilley, editors, Springer, 2007.

The moieties described below can be substituted or unsubstituted. “Substituted” refers to replacement of a hydrogen atom of a molecule or an R-group with one or more additional R-groups such as halogen, alkyl, haloalkyl, alkenyl, alkoxy, alkoxyalkyl, alkylthio, trifluoromethyl, acyloxy, hydroxy, hydroxyalkyl, mercapto, carboxy, cyano, acyl, aryloxy, aryl, arylalkyl, heteroaryl, amino, aminoalkyl, alkylamino, dialkylamino, morpholino, piperidino, pyrrolidin-1-yl, piperazin-1-yl, nitro, phosphine, phosphinate, phosphonate, sulfato, ═O, ═S, or other R-groups. Unless otherwise indicated, an optionally substituted group may have a substituent at each substitutable position of a group. Combinations of substituents contemplated herein are preferably those that result in the formation of stable (e.g., not substantially altered for a week or longer when kept at a temperature of 40° C. or lower in the absence of moisture or other chemically reactive conditions), or chemically feasible, compounds.

“Hydroxy”, “thiol”, “cyano”, “nitro”, and “formyl” refer, respectively, to —OH, —SH, CN, —NO₂, and —CHO.

“Acyl” refers to a RC(═O)— radical, wherein R is alkyl, cycloalkyl, aryl, heteroalkyl, heteroaryl, or heterocycloalkyl, which are as described herein. In some embodiments, it is a C₁-C₁₂ acyl radical, which refers to the total number of chain or ring atoms of the alkyl, cycloalkyl, aryl, heteroalkyl, heteroaryl, or heterocycloalkyl portion of the acyloxy group plus the carbonyl carbon of acyl, i.e., the other ring or chain atoms plus carbonyl. If the R radical is heteroaryl or heterocycloalkyl, the hetero ring or chain atoms contribute to the total number of chain or ring atoms.

“Acyloxy” refers to a RC(═O)O— radical, wherein R is alkyl, cycloalkyl, aryl, heteroalkyl, heteroaryl, or heterocycloalkyl, which are as described herein. In some embodiments, it is a C₁-C₄ acyloxy radical, which refers to the total number of chain or ring atoms of the alkyl, cycloalkyl, aryl, heteroalkyl, heteroaryl, or heterocycloalkyl portion of the acyloxy group plus the carbonyl carbon of acyl, i.e., the other ring or chain atoms plus carbonyl. If the R radical is heteroaryl or heterocycloalkyl, the hetero ring or chain atoms contribute to the total number of chain or ring atoms.

“Alkyl” refers to a group of 1-18, 1-16, 1-12, 1-10, preferably 1-8, more preferably 1-6 unsubstituted or substituted hydrogen-saturated carbons connected in linear, branched, or cyclic fashion, including the combination in linear, branched, and cyclic connectivity. Non-limiting examples include methyl, ethyl, propyl, isopropyl, butyl, and pentyl.

“Cycloalkyl” refers to a monocyclic or polycyclic non-aromatic radical that contains carbon and hydrogen, and may be saturated, or partially unsaturated. Cycloalkyl groups include groups having from 3 to 10 ring atoms (e.g., C₃-C₁₀ cycloalkyl) or groups having two rings, each ring having from 4 to 12 ring atoms (e.g., C₄-C₁₂ bicycloalkyl). Wherever it appears herein, a numerical range such as “3 to 10” refers to each integer in the given range; e.g., “3 to 10 carbon atoms” means that the cycloalkyl group may consist of 3 carbon ring atoms, 4 carbon ring atoms, 5 carbon ring atoms, etc., up to and including 10 carbon ring atoms. In some embodiments, it is a C₃-C₈ cycloalkyl radical. In some embodiments, it is a C₃-C₅ cycloalkyl radical. Examples of cycloalkyl groups include, but are not limited to, cyclopropyl, cyclobutyl, cyclopentyl, cyclopentenyl, cyclohexyl, cyclohexenyl, cycloseptyl, cyclooctyl, cyclononyl, cyclodecyl, and norbornyl. The term “cycloalkyl” also refers to spiral ring systems, in which the cycloalkyl rings share one carbon atom.

“Heterocycloalkyl” refers to a 3- to 18-membered nonaromatic ring (e.g., C₃-C₁₈ heterocycloalkyl) radical that comprises two to twelve ring carbon atoms and from one to six heteroatoms selected from nitrogen, oxygen, and sulfur. Whenever it appears herein, a numerical range such as “3 to 18” refers to each integer in the given range; e.g., “3 to 18 ring atoms” means that the heterocycloalkyl group may consist of 3 ring atoms, 4 ring atoms, etc., up to and including 18 ring atoms. In some embodiments, it is a C₅-C₁₀ heterocycloalkyl. In some embodiments, it is a C₄-C₁₀ heterocycloalkyl. In some embodiments, it is a C₃-C₁₀ heterocycloalkyl. The heterocycloalkyl radical may be a monocyclic, bicyclic, tricyclic, or tetracyclic ring system, which may include fused or bridged ring systems. The heteroatoms in the heterocycloalkyl radical may be optionally oxidized. One or more nitrogen atoms, if present, may optionally be quaternized. The heterocycloalkyl radical may be partially or fully saturated. The heterocycloalkyl may be attached to the rest of the molecule through any atom of the ring(s). Examples of such heterocycloalkyl radicals include, but are not limited to, 6,7-dihydro-5H-cyclopenta[b]pyridine, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. In some embodiments, the heterocycloalkyl group is aziridinyl, azetidinyl, pyrrolidinyl, piperidinyl, homopiperidinyl, morpholinyl, thiomorpholinyl, piperazinyl, tetrahydrofuranyl, tetrahydrothienyl, dihydrooxazolyl, tetrahydropyranyl, tetrahydrothiopyranyl, indolinyl, tetrahydroquinolyl, tetrahydroisoquinolin, and benzoxazinyl, preferably dihydrooxazolyl and tetrahydrofuranyl.

“Halo” refers to any of halogen atoms fluorine (F), chlorine (Cl), bromine (Br), or iodine (I). Examples of such halo groups can be fluorine.

“Haloalkyl” refers to an alkyl substituted by one or more halo(s).

“Alkenyl” refers to a group of unsubstituted or substituted hydrocarbons containing 2-18, 2-16, 2-12, 2-10, preferably 2-8, more preferably 2-6 carbons, which are linear, branched, cyclic, or in combination thereof, with at least one carbon-to-carbon double bond.

“Haloalkenyl” refers to an alkenyl substituted by one or more halo(s).

“Alkynyl” refers to a group of unsubstituted or substituted hydrocarbons containing 2-18, 2-16, 2-12, 2-10, preferably 2-8, more preferably 2-6 carbons, which are linear, branched, cyclic, or in combination thereof, with at least one carbon-to-carbon triple bond.

“Haloalkynyl” refers to an alkynyl substituted by one or more halo(s).

“Amino protecting group” refers to those groups intended to protect an amino group against undesirable reactions during synthetic procedures and which can later be removed to reveal the amine. Commonly used amino protecting groups are disclosed in Protective Groups in Organic Synthesis, Greene, T. W.; Wuts, P. G. M., John Wiley & Sons, New York, N.Y., (3rd Edition, 1999). Amino protecting groups include acyl groups such as formyl, acetyl, propionyl, pivaloyl, t-butylacetyl, 2-chloroacetyl, 2-bromoacetyl, trifluoroacetyl, trichloroacetyl, o-nitrophenoxyacetyl, alpha-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxy-carbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, alpha,alpha-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethylsilylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyloxycarbonyl, phenylthiocarbonyl and the like; aralkyl groups such as benzyl, triphenylmethyl, benzyloxymethyl and the like; and silyl groups such as trimethylsilyl and the like. Amine protecting groups also include cyclic amino protecting groups such as phthaloyl and dithiosuccinimidyl, which incorporate the amino nitrogen into a heterocycle. Typically, amino protecting groups include formyl, acetyl, benzoyl, pivaloyl, t-butylacetyl, phenylsulfonyl, Alloc, Teoc, benzyl, Fmoc, Boc, and Cbz.

“Amino” refers to amino and substituted amino groups, for example, primary amines, secondary amines, tertiary amines, and quaternary amines. Specifically, “amino” refers to NRₐR_{b}, wherein Rₐ and R_{b}, both directly connected to the N, can be independently selected from hydrogen, deuterium, halo, hydroxy, cyano, formyl, nitro, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, a nitrogen protective group, —(CO)-alkyl, —(CO)—O-alkyl, or —S(O)ₙR_{c} (n=0 to 2, R_{c} is directly connected to S), wherein R_{c} is independently selected from hydrogen, deuterium, halo, amino, hydroxy, thiol, cyano, formyl, alkyl, haloalkyl, alkenyl, haloalkenyl, alkynyl, haloalkynyl, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

An “ammonium” can be a quaternary amine, for example, a cation of primary amine, secondary amine, tertiary amines or quaternary amine. For example, an ammonium can be a cation of an alkyl amine, such as an alkoxyalkyl amine, e.g., tris(hydroxymethyl)aminomethane or meglumine (methylglucamine).

“Aryl” refers to a C₆-C₁₄ aromatic hydrocarbon. For example, aryl can be phenyl, napthyl, or fluorenyl.

“Heteroaryl” refers to a C₅-C₁₄ aromatic hydrocarbon having one or more heteroatoms, such as N, O, or S. The heteroaryl can be substituted or unsubstituted. Examples of a heteroaryl include, but are not limited to, azaindole, azepinyl, acridinyl, benzimidazolyl, benzindolyl, 1,3-benzodioxolyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzofurazanyl, benzothiazolyl, benzothienyl, benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furazanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyranyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, thiapyranyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). In some embodiments, the heteroaryl can be dithiazinyl, furyl, imidazolyl, azaindolyl, indolyl, isoquinolinyl, isoxazolyl, oxadiazolyl (e.g., (1,3,4)-oxadiazolyl, (1,2,3)-oxadiazolyl, or (1,2,4)-oxadiazolyl, oxazolyl, pyrazinyl, pyrazolyl, pyrazyl, pyridazinyl, pyridinyl, pyrimidinyl, pyrimidyl, pyrrolyl, quinolinyl, tetrazolyl, thiazolyl, thienyl, triazinyl, (1,2,3)-triazolyl, or (1,2,4)-triazolyl. The substituent on the heteroaryl group can be amino, alkylamino, or methyleneamino.

“Carbocycle” refers to a C₃-C₁₄ cyclic hydrocarbon. For example, aryl can be phenyl, napthyl, or fluorenyl.

“Heterocycle” refers to a C₅-C₁₄ cyclic hydrocarbon having one or more heteroatoms, such as N, O, or S.

“Alkoxy” refers to an alkyl connected to an oxygen atom (—O-alkyl).

“Haloalkoxy” refers to a haloalkyl connected to an oxygen atom (—O-haloalkyl).

“Thioalkoxy” refers to an alkyl connected to a sulfur atom (—S-alkyl).

“Halothioalkoxy” refers to a haloalkyl connected to a sulfur atom (—S-haloalkyl).

“Carbonyl” refers to —(CO)—, wherein (CO) indicates that the oxygen is connected to the carbon with a double bond.

“Alkanoyl (or acyl)” refers to an alkyl connected to a carbonyl group [—(CO)-alkyl].

“Haloalkanoyl” or “haloacyl” refers to a haloalkyl connected to a carbonyl group [—CO)-haloalkyl].

“Thiocarbonyl” refers to —(CS)—, wherein (CS) indicates that the sulfur is connected to the carbon with a double bond.

“Thioalkanoyl (or thioacyl)” refers to an alkyl connected to a thiocarbonyl group [—(CS)-alkyl].

“Halothioalkanoyl” or “halothioacyl” refers to a haloalkyl connected to a thiocarbonyl group [—(CS)-haloalkyl].

“Carbonyloxy” refers to an alkanoyl (or acyl) connected to an oxygen atom [—O—(CO)-alkyl].

“Halocarbonyloxy” refers to a haloalkanoyl (or haloacyl) connected to an oxygen atom [—O—(CO)-haloalkyl].

“Carbonylthio” refers to an alkanoyl (or acyl) connected to a sulfur atom [—S—(CO)-alkyl].

“Halocarbonylthio” refers to a haloalkanoyl (or haloacyl) connected to a sulfur atom [—S—(CO)-haloalkyl].

“Thiocarbonyloxy” refers to a thioalkanoyl (or thioacyl) connected to an oxygen atom [—O—(CS)-alkyl].

“Halothiocarbonyloxy” refers to a halothioalkanoyl (or halothioacyl) connected to an oxygen atom [—O—(CS)-haloalkyl].

“Thiocarbonylthio” refers to a thioalkanoyl (or thioacyl) connected to a sulfur atom [—S—(CS)-alkyl].

“Halothiocarbonylthio” refers to a halothioalkanoyl (or halothioacyl) connected to a sulfur atom [—S—(CS)-haloalkyl].

Phosphate Derivatives of Indole Compounds

An aspect of the present disclosure relates to phosphate derivatives of indole compounds. The indole compounds can bind specifically to and modulate human aryl hydrocarbon receptor (AhR). Without wishing to be bound by theory, it is contemplated that AhR bound by one of the indole compounds is agonized with respect to the receptor's immune-stimulatory activity. The indole compounds can be those described in U.S. Provisional Patent Application No. 62/717,387, filed Aug. 10, 2018, U.S. Provisional Patent Application No. 62/588,751, filed Nov. 20, 2017, and WO 2019/099977, each of which is incorporated by reference herein in its entirety.

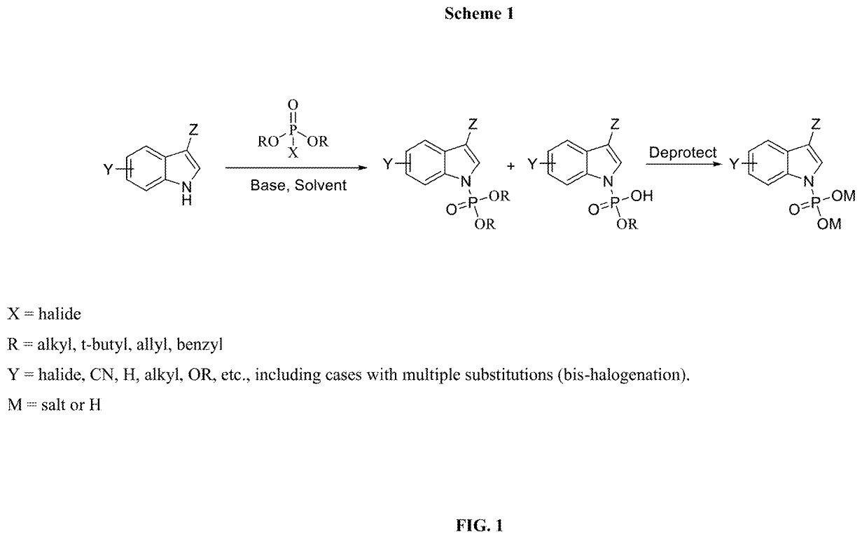

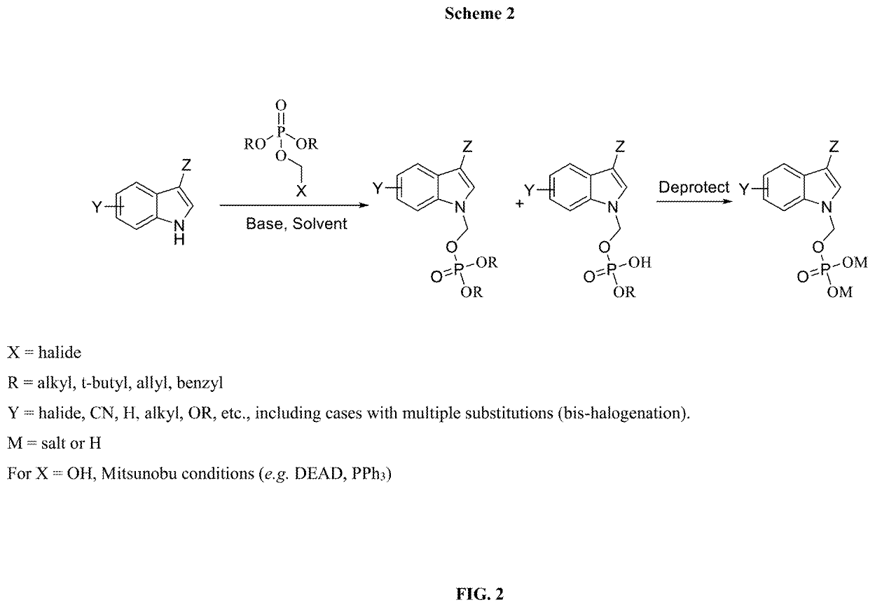

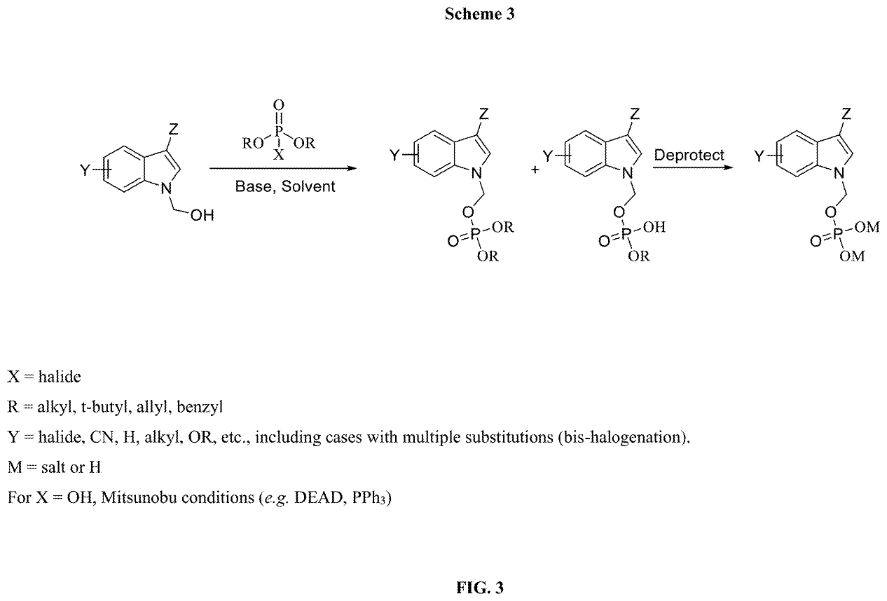

The phosphate derivatives can be prepared by the general synthetic schemes shown in FIGS. 1, 2, and 3.

The phosphate derivative of an indole compound can be a phosphate salt. The salt can be an alkali metal salt.

The compound can be an indolo-phosphoramidate analog (IPA). The indolo-phosphoramidate analog can have an N—P bond. In certain embodiments, the indolo-phosphoramidate analog, such as an indolo-methyleneoxyphosphate, can include a labile linker between the indole nitrogen and the phosphate phosphorus. The labile linker can be of the formula —(CR₂R₃—O)ₓ—, where x is 0, 1, 2, 3, 4, 5, or 6 and each of R₂ and R₃ can be, independently, H, or C₁-C₆ alkyl. In certain embodiments, x is 0 or 1. In certain embodiments, each of R₂ and R₃ can be, independently, H.

In one aspect, a compound can have Formula I:

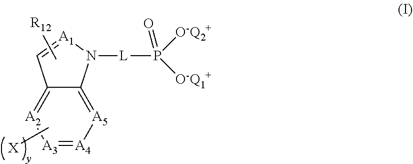

R₁₂ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Each of A₁, A₂, A₃, A₄, and A₅, independently, can be CR₂ or N.

L can be —(CR₂R₃—O)ₙ— or a bond.

R₂ can be H or C₁-C₆ alkyl, R₃ can be H or C₁-C₆ alkyl, or, together, R₂ and R₃ form a C₃-C₈ cycloalkyl.

n can be 0, 1, 2, 3, 4, 5, or 6.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Q₁⁺ and Q₂⁺ can be each, independently, a monocation, or together can be a dication or one or both of Q₁⁺ or Q₂⁺ can be H, C₁-C₆ alkyl, benzyl, allyl, or —(CR₂R₃—O)—R₂₃, and R₂₃ can be H or C₁-C₆ alkyl. The alkyl can be a substituted alkyl, for example an alkoxy alkyl, amino alkyl, alkyl ester, alkyl carbamate, or alkyl carbonate.

In another aspect, a compound can have a structure of Formula II:

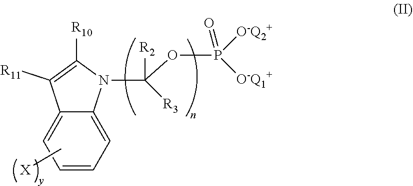

R₁₀ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

R₁₁ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

One of R₁₀ and Ru is H or C₁-C₆ alkyl.

R₂ can be H or C₁-C₆ alkyl, R₃ can be H or C₁-C₆ alkyl, or, together, R₂ and R₃ can form a C₃-C₈ cycloalkyl.

y can be 0, 1, 2, 3, or 4.

Each X, independently, can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

Q₁⁺ and Q₂⁺ can be each, independently, a monocation, or together are a dication.

n can be 0, 1, 2, 3, 4, 5, or 6. For example, n can be 0 or n can be 1.

In certain circumstances, the compound can be of Formula III:

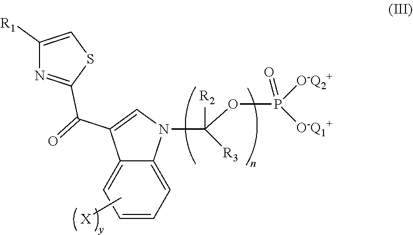

wherein:

R₁ can be —C(═O)—R₄, cyano, an oxadiazole, or a thiadiazole.

R₂ and R₃ can be each, independently, hydrogen, or C₁-C₆ alkyl.

R₄ can be selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and —S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio.

In certain circumstances, the compound can be of Formula IV:

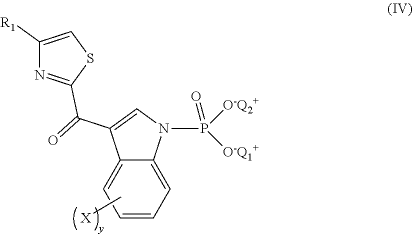

wherein:

R₁ can be —C(═O)—R₄, cyano, an oxadiazole, or a thiadiazole.

R₄ can be selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio.

Each X, independently, can be H or halogen.

In certain circumstances, the compound can be of Formula V:

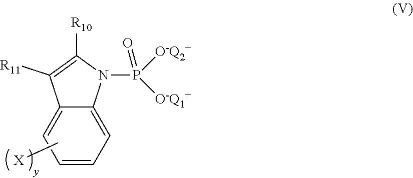

wherein:
R₁₀ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.
R₁₁ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ can be H or C₁-C₆ alkyl.
y can be 0, 1, 2, 3, or 4.
Each X, independently, can be H or halogen.

In certain circumstances, Q₁⁺ and Q₂⁺ can be each, independently, H or an alkali metal.

In certain circumstances, Q₁⁺ and Q₂⁺ can be each, independently, selected from the group consisting of lithium, sodium, and potassium.

In certain circumstances, Q₁⁺ and Q₂⁺ can be each, independently, selected from the group consisting of ammonium and alkyl ammonium. For example, the alkyl ammonium can be a hydroxyalkyl ammonium.

In certain circumstances, Q₁⁺ and Q₂⁺ together can be an alkaline earth metal salt.

In certain circumstances, Q₁⁺ and Q₂⁺ can be each independently selected from the group consisting of zinc, calcium, and magnesium.

In certain circumstances, R₁ can be —C(═O)—R₄, and R₄ is C₁-C₆ alkyl or C₁-C₆ alkoxy.

In certain circumstances, R₁ can be an oxadiazole or a thiadiazole. The oxadiazole or thiadiazole can be substituted, for example, with a C₁-C₆ alkyl, haloalkyl, halo, amino, or hydroxy. The oxadiazole or thiadiazole can be a 1,3,4, 1,2,4 or 1,2,3 heterocycle.

In certain circumstances, n can be 0, 1, or 2.

In certain circumstances, Q₁⁺ and Q₂⁺ can be each, independently, lithium, sodium, or potassium, y can be 0, 1, or 2, and X can be F, Cl, or Br.

In certain circumstances, the compound can be selected from the group consisting of:

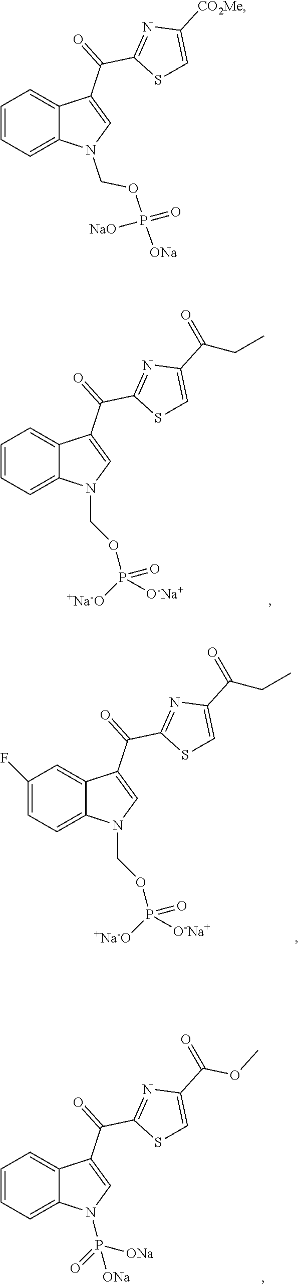

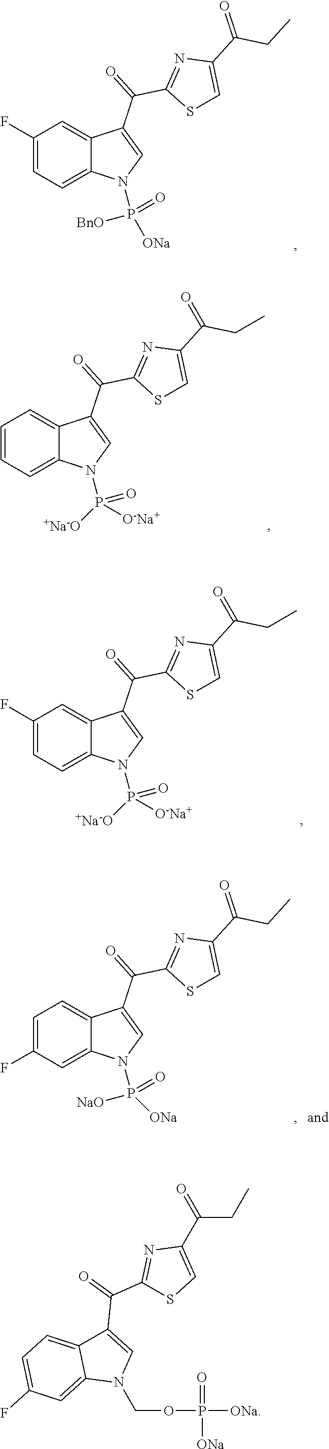

In certain circumstances, R₁ can be an unsubstituted or substituted oxadiazole.

In certain circumstances, n can be 0.

In certain circumstances, Q₁⁺ and Q₂⁺ each can be lithium, sodium, or potassium.

In certain circumstances, the compound can be selected from the group consisting of:

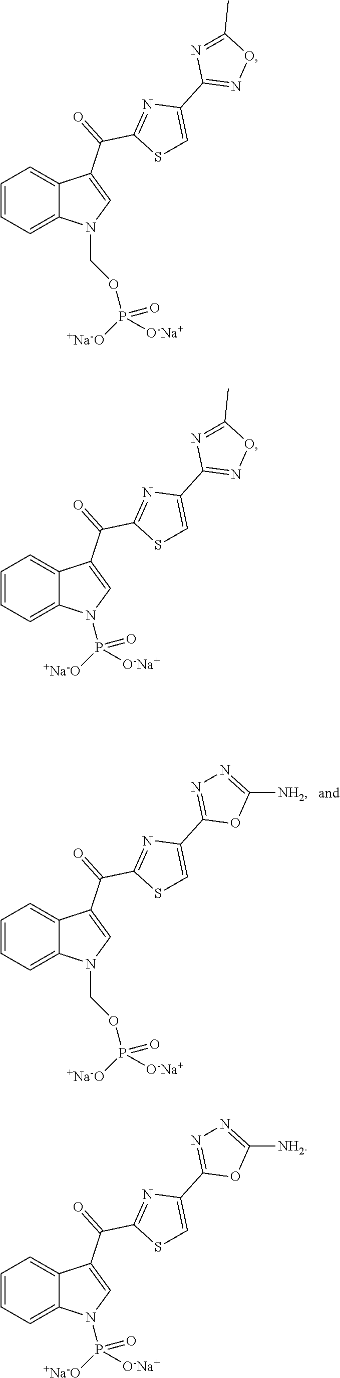

In certain circumstances, a compound can be of Formula V:

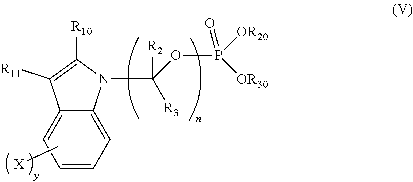

R₁₀ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio.

R₁₁ can be hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ can be H or C₁-C₆ alkyl.

R₂ can be H or C₁-C₆ alkyl, R₃ can be H or C₁-C₆ alkyl, or, together, R₂ and R₃ can form a C₃-C₈ cycloalkyl.

y can be 0, 1, 2, 3, or 4.

Each X, independently, can be H or halogen.

R₂₀ and R₃₀ each, independently, can be H, C₁-C₆ alkyl, allyl, or benzyl, or one of R₂₀ or R₃₀ is H, C₁-C₆ alkyl, allyl, or benzyl and the other of R₂₀ or R₃₀ is a cation.

n can be 0, 1, 2, 3, 4, 5, or 6. Preferably, n can be 0 or 1.

Exemplary compounds are shown in Table 1.

TABLE 1
Representative phosphate derivatives of indole compounds

| Compound | Structural Formula |
|---|---|
| A (ARI-010) | 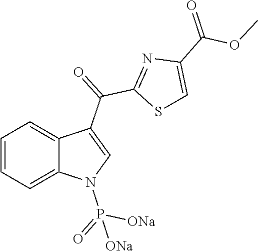 |
| B (ARI-012) | 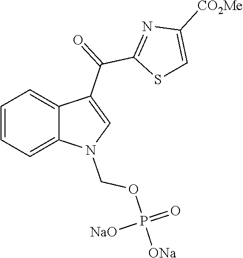 |
| C (ARI-115) | 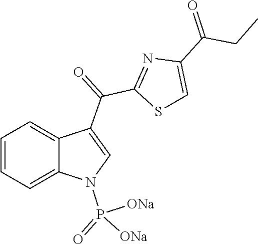 |
| D | 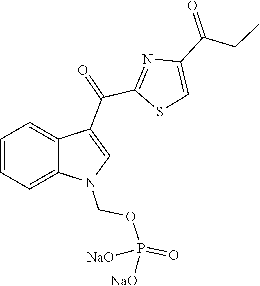 |
| E | 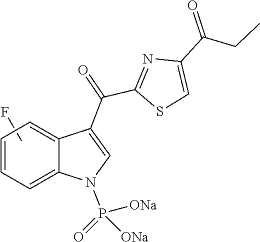 |
| F | 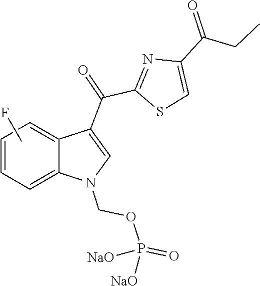 |
| G | 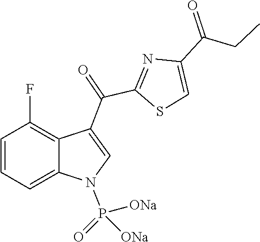 |
| H | 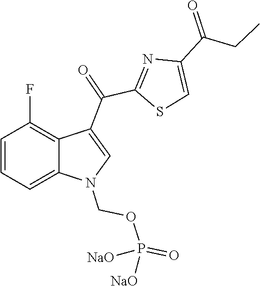 |
| I (ARI-152) | 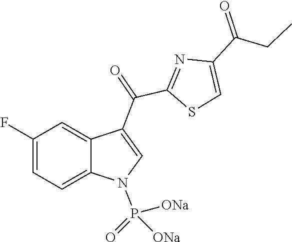 |
| J (ARI-155) | 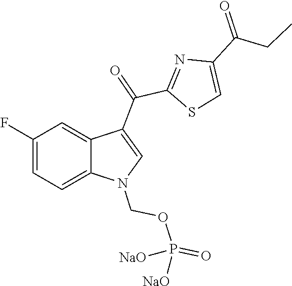 |
| K (ARI-158) | 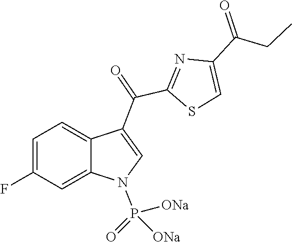 |
| L (ARI-160) | 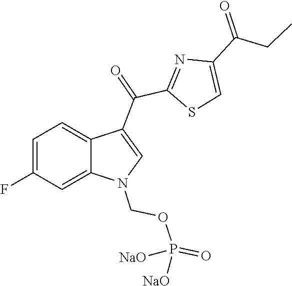 |
| M | 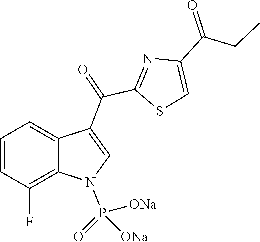 |
| N | 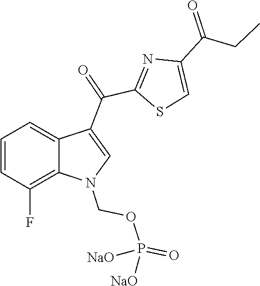 |
| O | 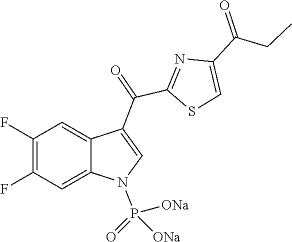 |
| P | 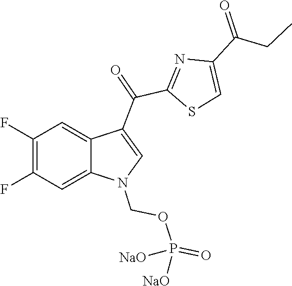 |
| Q | 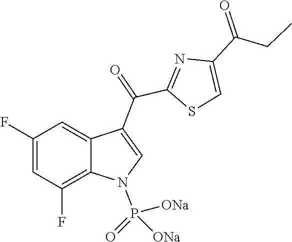 |
| R | 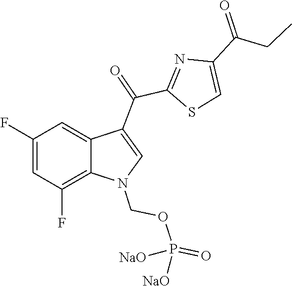 |
| S | 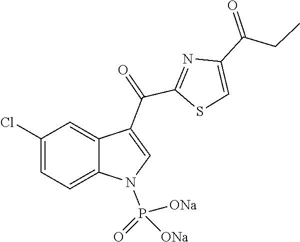 |
| T | 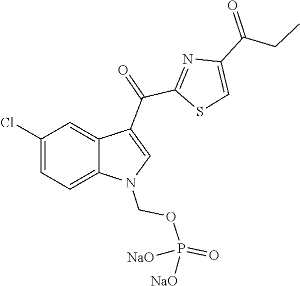 |
| U | 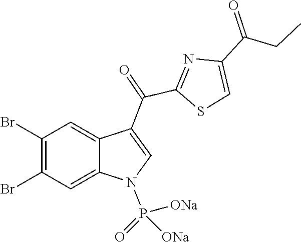 |
| V | 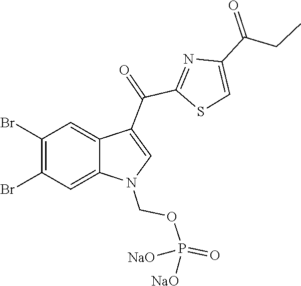 |
| W (ARI-151) | 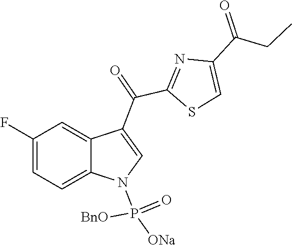 |
| X | 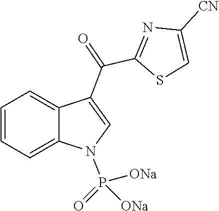 |
| Y | 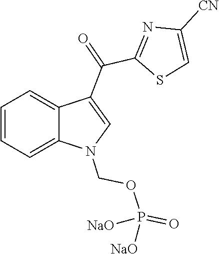 |
| Z | 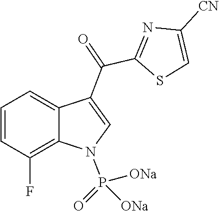 |
| AA | 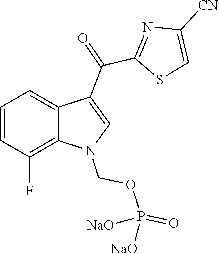 |
| AB | 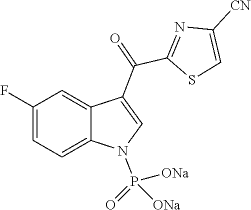 |
| AC | 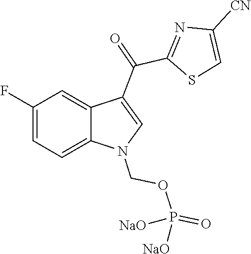 |
| AD | 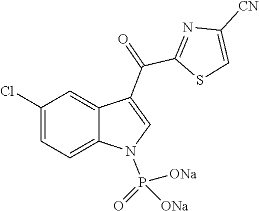 |
| AE | 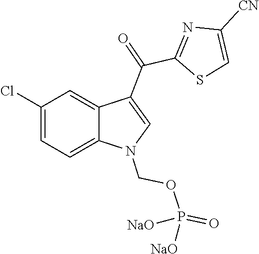 |
| AF (ARI-098) | 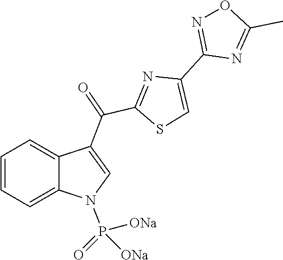 |
| AG | 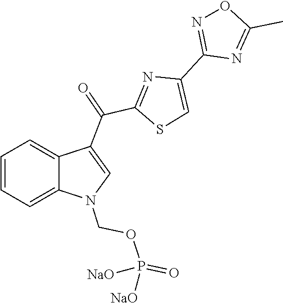 |
| AH | 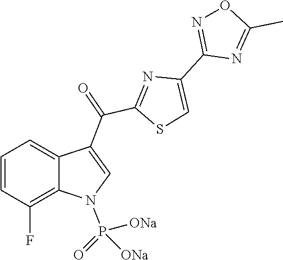 |
| AI | 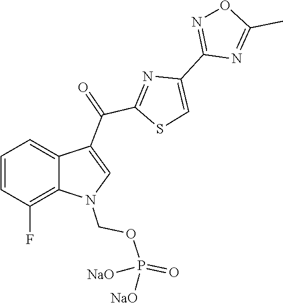 |
| AJ | 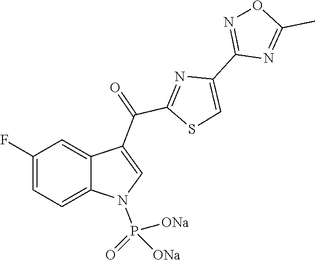 |
| AK | 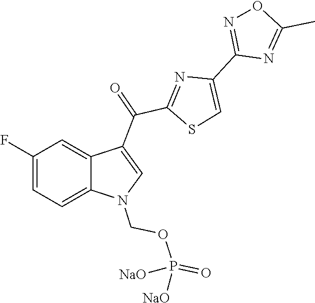 |
| AL | 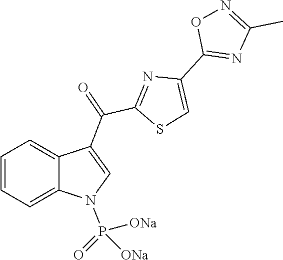 |
| AM | 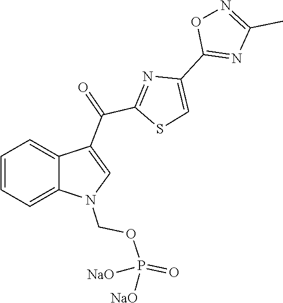 |
| AN | 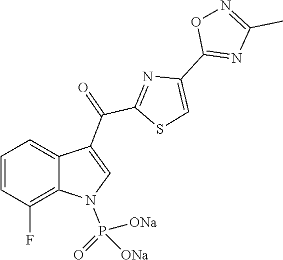 |
| AO | 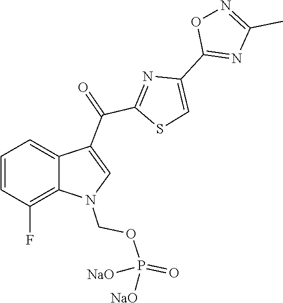 |
| AP | 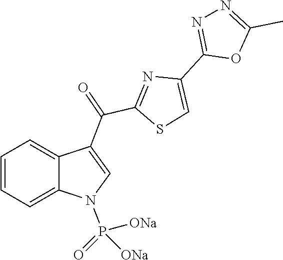 |
| AQ | 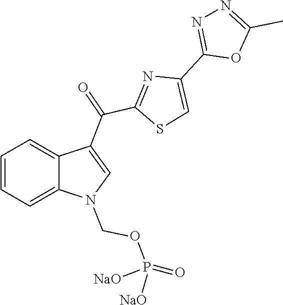 |
| AR (ARI-153) | 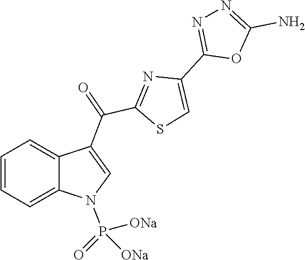 |
| AS | 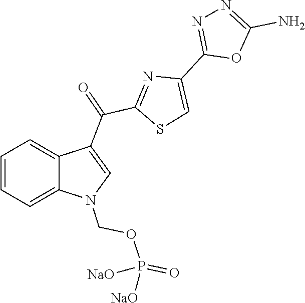 |
| AT (ARI-159) | 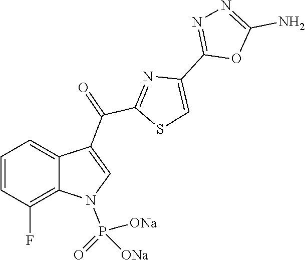 |
| AU (ARI-166) | 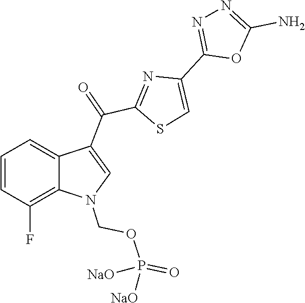 |
| AV | 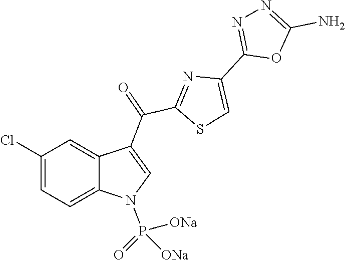 |
| AW | 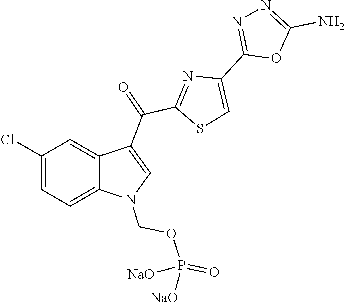 |
| AX | 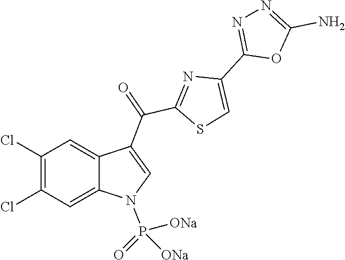 |
| AY | 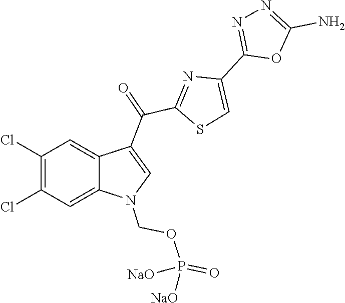 |
| AZ | 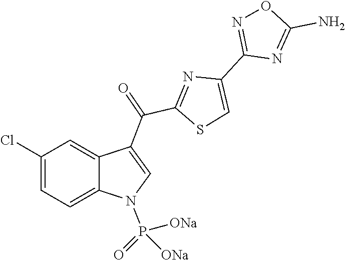 |
| BA | 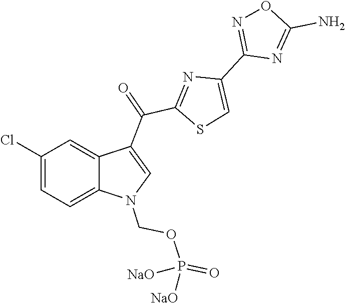 |
| BB | 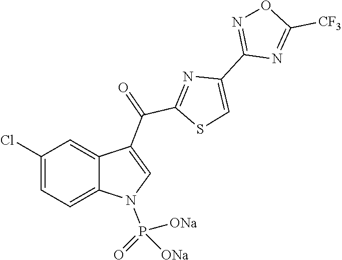 |
| BC | 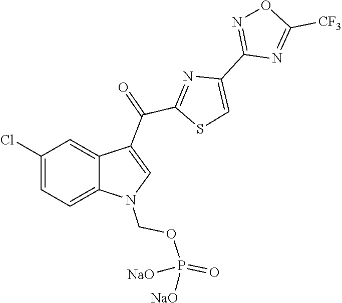 |
| BD | 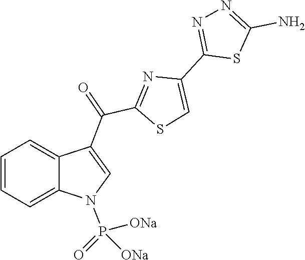 |
| BE | 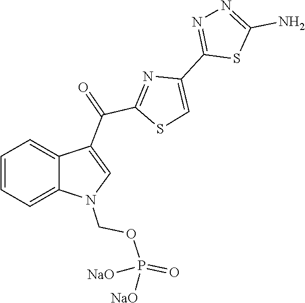 |
| BF | 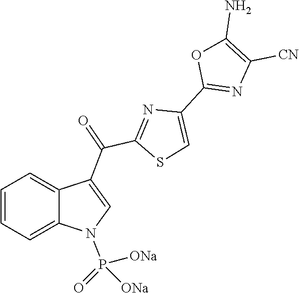 |
| BG | 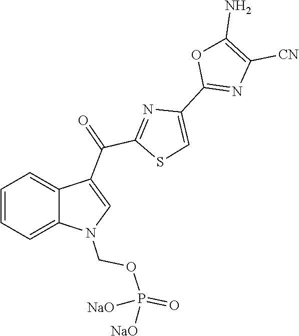 |
| BH | 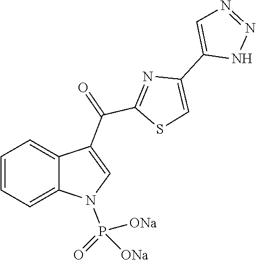 |
| BI | 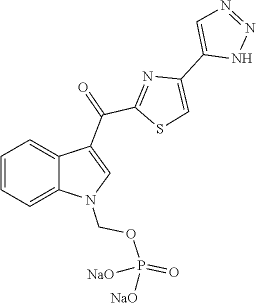 |
| BJ | 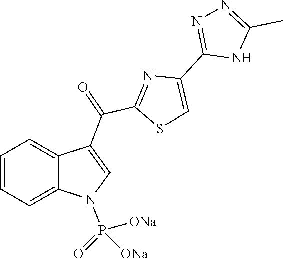 |
| BK | 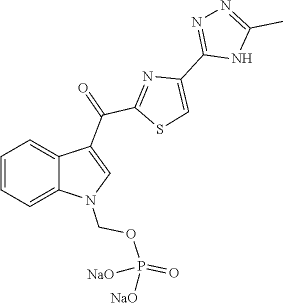 |
| BL | 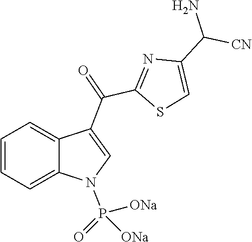 |
| BM | 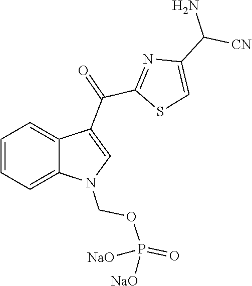 |
| BN | 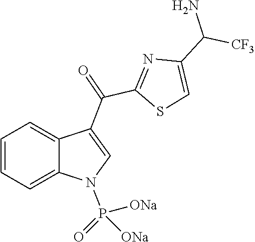 |
| BO | 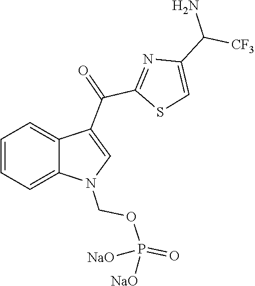 |
| BP | 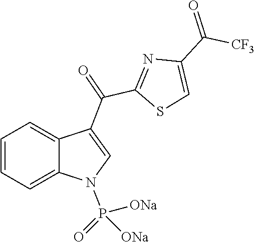 |
| BQ | 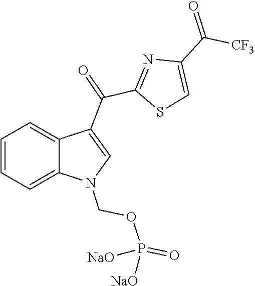 |
| BR | 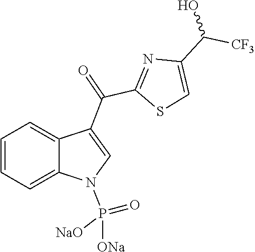 |
| BS | 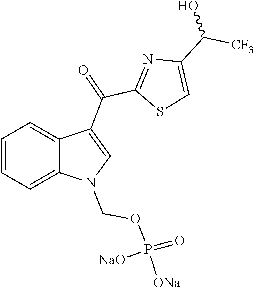 |

Compound A had the following spectroscopic characteristics: LC/MS ESI MS m/z 365 [M−H]⁻, ¹H NMR (500 MHz, D₂O) δ 9.01 (d, J=3.5 Hz, 1H), 8.66 (s, 1H), 8.22 (dd, J=6.5, 2.0 Hz, 1H), 7.96 (dd, J=6.0, 2.5 Hz, 1H), 7.39-7.33 (m, 2H), 3.93 (s, 3H).

Compound B had the following spectroscopic characteristics: LC/MS ESI MS m/z 395 [M−H]⁻, ¹H NMR (500 MHz, D₂O) δ 9.05 (d, J=3.5 Hz, 1H), 8.67-8.65 (m, 1H), 8.28-8.25 (m, 1H), 7.79 (d, J=8.0, Hz, 1H), 7.45-7.39 (m, 2H), 5.88 (dd, J=6.5, 2.5 Hz, 2H), 3.94 (s, 3H).

Compound C had the following spectroscopic characteristics: LC/MS ESI MS m/z 363 [M−H]⁻, ESI MS m/z 365 [M+H]⁺, ¹H NMR (500 MHz, D₂O) δ 9.13 (d, J=3.5 Hz, 1H), 8.66 (s, 1H), 8.25-8.24 (m, 1H), 7.97-7.95 (m, 1H), 7.38-7.34 (m, 2H), 3.23 (q, J=7.0 Hz, 2H), 1.15 (t, J=7.0 Hz, 3H). ³¹P NMR (202 MHz, D₂O) δ −4.56.

Compound I had the following spectroscopic characteristics: LC/MS ESI MS m/z 381 [M-N]⁻, ¹H NMR (500 MHz, D₂O) δ 9.13 (d, J=3.5 Hz, 1H), 8.62 (s, 1H), 7.92-7.88 (m, 2H), 7.11 (dt, J=9.5, 3.0 Hz, 1H), 3.21 (q, J=7.0 Hz, 2H), 1.15 (t, J=7.0 Hz, 3H); ¹⁹F NMR (470 MHz, D₂O) δ −120.60; ³¹P NMR (202 MHz, D₂O) δ −4.55.

Compound W had the following spectroscopic characteristics: LC/MS ESI MS m/z 471 [M−H]⁻, ¹H NMR (500 MHz, D₂O) δ 8.72 (d, J=3.5 Hz, 1H), 8.00 (s, 1H), 7.61 (dd, J=9.0, 4.5 Hz, 1H), 7.44 (dd, J=9.5, 2.0 Hz, 1H), 6.96 (dt, J=9.0, 2.5 Hz, 1H), 6.91-6.82 (m, 5H), 4.77 (d, J=11.0 Hz, 2H), 2.97 (q, J=7.0 Hz, 2H), 1.07 (t, J=7.5 Hz, 3H); ¹⁹F NMR (470 MHz, D₂O) δ −119.99; ³¹P NMR (202 MHz, D₂O) δ −8.19.

Compound AF had the following spectroscopic characteristics: LC/MS ESI MS m/z 390 [M−H]⁻, ¹H NMR (500 MHz, D₂O) δ 9.15 (d, J=3.5 Hz, 1H), 8.38 (d, J=0.5 Hz, 1H), 8.24-8.22 (m, 1H), 7.96-7.95 (m, 1H), 7.38-7.33 (m, 2H); ³¹P NMR (202 MHz, D₂O) δ −4.67.

Compound AQ had the following spectroscopic characteristics: LC/MS ESI MS m/z 391 [M+H]⁺, ¹H NMR (500 MHz, D₂O) δ 9.18 (d, J=4.0 Hz, 1H), 8.52 (s, 1H), 8.27-8.25 (m, 1H), 7.97-7.95 (m, 1H), 7.39-7.34 (m, 2H), 2.66 (s, 3H). ³¹P NMR (202 MHz, D₂O) δ −4.60.

Single stereochemical isomers, enantiomers, diastereomers, and pharmaceutically acceptable salts of the above exemplified compounds are also within the scope of the present disclosure. Pharmaceutically acceptable salts may be, for example, derived from suitable inorganic and organic acids and bases.

Acid addition salts can be prepared by reacting the purified compound in its free-based form with a suitable organic or inorganic acid and isolating the salt thus formed. Examples of pharmaceutically acceptable acid addition salts include, without limitations, salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid.

Base addition salts can be prepared by reacting the purified compound in its acid form with a suitable organic or inorganic base and isolating the salt thus formed. Such salts include, without limitations, alkali metal (e.g., sodium, lithium, and potassium), alkaline earth metal (e.g., magnesium and calcium), ammonium, alkylammonium, substituted alkylammonium and N⁺(C₁₋₄alkyl)₄ salts. The alkyl can be a hydroxyalkyl.

Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, and valerate salts.

The compounds of the present disclosure may be synthesized by methods known in the art or by methods illustrated in the Examples below.

Pharmaceutical Compositions and Use

An aspect of the present disclosure relates to pharmaceutical compositions comprising one or more compounds disclosed herein formulated with one or more pharmaceutically acceptable excipients or carriers (carrier system). The carrier system may include, for example, solvents, diluents, or other liquid vehicle, dispersion or suspension aids, surface active agents, isotonic agents, thickening or emulsifying agents, preservatives, fillers, extenders, disintegrating agents, solid binders, absorbents, lubricants, wetting agents, and the like. The pharmaceutical compositions can be administered to patients, for example, orally, or parenterally (e.g., subcutaneously, intravenously, or intramuscularly), intranasally, or topically. The pharmaceutical compositions may be provided, for example, in a form of cream, capsules, tablets, lozenges, or injectables.

Another aspect of the present disclosure relates to a method of stimulating the immune system in a patient in need thereof. The method includes administering to the patient a therapeutically effective amount of one or a combination of the compounds described herein. In some embodiments, the patient has an increased count of white blood cells, T and/or B lymphocytes, macrophases, neutrophils, natural killer (NK) cells, and/or platelets after the administering step. The patient may have cancer or may be immune compromised.

Accordingly, the present disclosure provides a method of treating cancer in a patient in need thereof, comprising administering to the patient a therapeutically effective amount of one or a combination of the compounds described herein. In some embodiments, the patient has a liquid cancer (e.g., a hematological malignancy such as lymphoma, leukemia, and myeloma) or a solid tumor. In some embodiments, the patient has lung cancer (e.g., nonsmall cell lung cancer), ovarian cancer, cancer of the Fallopian tube, cervical cancer, breast cancer, skin cancer (e.g., melanoma), colorectal cancer, stomach cancer, pancreatic cancer, liver cancer, mesothelioma, kidney cancer (e.g., renal cell carcinoma), bladder cancer, prostate cancer, soft tissue cancer, squamous cell carcinoma, head and neck cancer, glioma, or brain tumor. This is by no means to limit the therapeutic scope of the compounds, given their broad cancer inhibition capabilities. In some embodiments, the cancer is metastatic or otherwise advanced.

“Treat”, “treating”, and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment. Treatment of cancer encompasses inhibiting cancer growth (including causing partial or complete cancer regression), inhibiting cancer progression or metastasis, preventing cancer recurrence or residual disease, and/or prolonging the patient's survival. A “therapeutically effective amount” is an amount of the medication that can achieve the desired curative, palliative, or prophylactic effect for the treated condition.

A therapeutically effective amount of a compound can vary within wide limits and may be determined in each particular case based on the specific compound(s) being administered, the route of administration, the condition being treated, as well as the patient being treated.

In some embodiments, an effective amount for tumor therapy may be measured by its ability to stabilize disease progression and/or ameliorate symptoms in a patient, and preferably to reverse disease progression, e.g., by reducing tumor size. In some embodiments, a maintenance dosing may be provided after the patient is free of cancer to ensure its complete elimination or eradication, or prevention of residual disease. The duration of the maintenance dosing can be determined based on clinical trial data.

In some embodiments, a compound may be administered in combination with one or more other cancer therapeutic agents that also target AhR or target molecules other than AhR. Compounds can be formulated either separately from, or together with, the other cancer therapeutic agents. Compounds can be administered either at the same schedule as, or at a different schedule from, the other cancer therapeutic agents. The proportion of a compound relative to other cancer therapeutic agents may be determined by clinical trials. Combining the compounds with the other cancer therapeutic agents may further enhance the efficacy of one another. For example, a compound of the present invention can be administered with an immune checkpoint inhibitor, such as an inhibitor of PD-1, PD-L1 or PD-L2 (e.g., pembrolizumab, nivolumab, or atezolizumab), or administered with CAR-T therapy (e.g., axicabtagene ciloleucel), to achieve additive or synergistic anti-cancer effect.

Dosage regimens may be adjusted to provide the optimum desired response. Dosage unit form, as used herein, refers to physically discrete units suited as unitary dosages for the patients/subjects to be treated; each unit containing a predetermined quantity of compound calculated to produce the desired therapeutic effect in association with the required pharmaceutical carrier.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of this invention may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens are well-known in the relevant art and would be understood to be encompassed by the skilled artisan once provided the teachings disclosed herein.

It is contemplated that a suitable dose of a compound of the present invention may be in the range of 0.1-100 mg/kg, such as about 0.5-50 mg/kg, e.g., about 1-20 mg/kg. The compound may for example be administered in a dosage of at least 0.25 mg/kg, e.g., at least 0.5 mg/kg, such as at least 1 mg/kg, e.g., at least 1.5 mg/kg, such as at least 2 mg/kg, e.g., at least 3 mg/kg, such as at least 4 mg/kg, e.g., at least 5 mg/kg; and e.g., up to at most 50 mg/kg, such as up to at the most 30 mg/kg, e.g., up to at the most 20 mg/kg, such as up to at the most 15 mg/kg. Administration will normally be repeated at suitable intervals, e.g., twice a day, thrice a day, once a day, once every week, once every two weeks, or once every three weeks, and for as long as deemed appropriate by the responsible doctor, who may optionally increase or decrease the dosage as necessary.

Unexpectedly, the phosphate derivatives of indole compounds are hydrolytically stable. Some of the exemplified compounds show only about 2-4% degradation after 2 weeks in buffer solutions at pH 2, pH 6, and pH 10.

Unless otherwise defined herein, scientific and technical terms used in connection with the present invention shall have the meanings that are commonly understood by those of ordinary skill in the art. Exemplary methods and materials are described below, although methods and materials similar or equivalent to those described herein can also be used in the practice or testing of the present invention. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

General Section:

The indole compounds can be those described in U.S. Provisional Patent Application No. 62/717,387, filed Aug. 10, 2018, and U.S. Provisional Patent Application No. 62/588,751, filed Nov. 20, 2017, and WO 2019/099977, each of which is incorporated by reference in its entirety.

Unless otherwise noted, reagents and solvents were used as received from commercial suppliers. Dibenzyl phosphorochloridate and di-tert-butyl phosphorochloridate were obtained according to the method by Pierce, William M.; Taylor, K. Grant; Waite, Leonard C. U.S. Pat. Appl. Publ. 20080221070, 11 Sep. 2008. All non-aqueous reactions were carried out under an atmosphere of dry nitrogen (unless otherwise noted). Proton nuclear magnetic resonance spectra were obtained on a Bruker Ascend 500 spectrometer at 500 MHz. Spectra are given in ppm (δ) and coupling constants, J values, are reported in hertz (Hz). Tetramethylsilane was used as an internal standard for proton nuclear magnetic resonance. ¹⁹F (470 MHz) and ³¹P NMR (202 MHz) spectra were taken with ¹H decoupling and chemical shifts were not corrected with an external standard. Mass spectra and LCMS analyses were obtained using a Shimadzu 2020 single quadrupole mass spectrometer (DUIS, UP-LCMS). NMR and MS data for Examples are presented in Table 1.

### Example 1: Preparation of sodium (3-(4-(methoxycarbonyl)thiazole-2-carbonyl)-1H-indol-1-yl)phosphonate

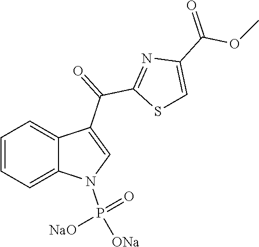

Step 1. Potassium hexamethyldisilazide (0.5 M in toluene) (13.97 ml, 6.99 mmol) was added to an ice-cold suspension of methyl 2-(1H-indole-3-carbonyl)thiazole-4-carboxylate (2 g, 6.99 mmol) in THF (34.9 ml). After 20 min, dibenzyl phosphorochloridate (0.112 g/mL in toluene) (23.29 ml, 6.99 mmol) was added slowly to the reaction mixture. After 1 hr, another 5 mL (1.5 mmol) of the dibenzyl phosphorochloridate solution was added. Upon completion, saturated NH₄Cl (aq) was added and the mixture was extracted twice with EtOAc. The combined organic phases were washed with brine, dried over Na₂SO₄, filtered, and concentrated to give methyl 2-(1-(bis(benzyloxy)phosphoryl)-1H-indole-3-carbonyl)thiazole-4-carboxylate as a brown solid (6.99 mmol). The crude material was taken on as is. ESI MS m/z 547 [M+H]⁺.

Step 2. MeOH (95 ml) was added to a mixture of crude methyl 2-(1-(bis(benzyloxy)phosphoryl)-1H-indole-3-carbonyl)thiazole-4-carboxylate (2.6 g, 4.76 mmol), 20% Pd(OH)₂ on carbon (3.34 g, 4.76 mmol), and sodium bicarbonate (1.199 g, 14.27 mmol). Next, nitrogen was sparged through the mixture followed by a hydrogen sparge using a balloon. The reaction was then stirred under a balloon of hydrogen. Upon completion, the solution was sparged with nitrogen for 15 min to remove excess hydrogen. Then, 20 mL of H₂O was added and the mixture was carefully filtered through a Celite pad. The pad was washed with 9:1 MeOH/H₂O and the filtrate was concentrated to dryness. The resulting yellow residue was treated with H₂O, briefly sonicated, and then filtered to remove solids. The aqueous filtrate was purified by reverse phase chromatography (C18, 5% CH₃CN/H₂O) to give sodium (3-(4-(methoxycarbonyl)thiazole-2-carbonyl)-1H-indol-1-yl)phosphonate (1.24 g, 63%) as a yellow solid after lyophilization.

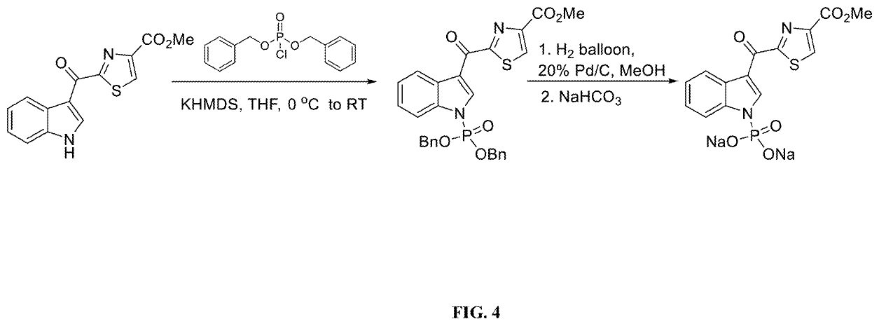

The synthetic approach is shown in FIG. 4.

### Example 2: Preparation of sodium (3-(4-(methoxycarbonyl)thiazole-2-carbonyl)-1H-indol-1-yl)methyl phosphate

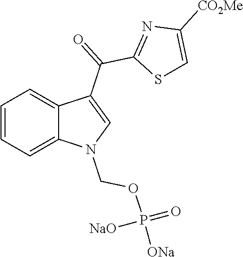

Step 1. Potassium hexamethyldisilazide (0.5 M in toluene) (2.494 mL, 1.247 mmol) was added to an ice-cold suspension of methyl 2-(1H-indole-3-carbonyl)thiazole-4-carboxylate (357 mg, 1.247 mmol) in THF (20 mL). After 20 min, dibenzyl (chloromethyl) phosphate (407 mg, 1.247 mmol) was added to the reaction mixture. Next, the cold bath was removed and the reaction was stirred overnight. Following day, silica gel was added and the mixture was concentrated to dryness. Chromatography (silica gel, CH₂Cl₂ to 6% MeOH/CH₂Cl₂) gave impure methyl 2-(1-(((bis(benzyloxy)phosphoryl)oxy)methyl)-1H-indole-3-carbonyl)thiazole-4-carboxylate (498 mg) as a yellow foam. The crude material was taken on as is. ESI MS m/z 577 [M+H]⁺.

Step 2. 20% Pd(OH)₂ on carbon (255 mg, 1.816 mmol) was added to a solution of crude methyl 2-(1-(((bis(benzyloxy)phosphoryl)oxy)methyl)-1H-indole-3-carbonyl)thiazole-4-carboxylate (498 mg, 0.864 mmol) in methanol (20 mL) and THF (5.00 mL). Next, a H₂ balloon was attached and the mixture was sparged briefly followed by overnight stirring under a H₂ balloon. Following day, the reaction mixture was sparged with nitrogen and then filtered through a Celite pad. The filtrate was then concentrated. The residue was dissolved in THF/H₂O (using a minimum of THF) and treated with saturated NaHCO₃ (aq) until basic. This mixture was purified by reverse phase chromatography (C18, H₂O to CH₃CN) to give sodium (3-(4-(methoxycarbonyl)thiazole-2-carbonyl)-1H-indol-1-yl)methyl phosphate (58 mg, 15%) after lyophilization from water/acetonitrile.

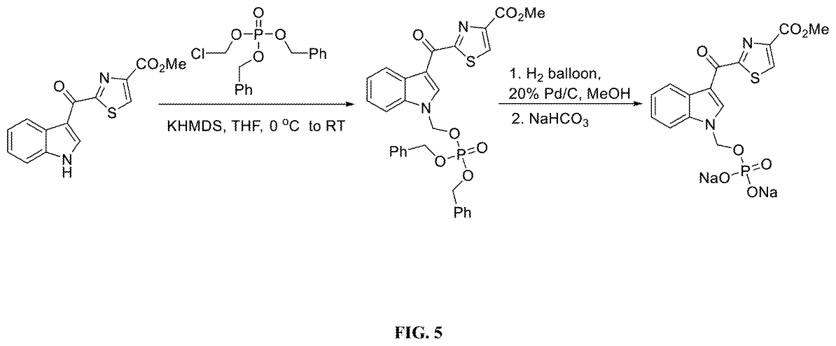

The synthetic approach is shown in FIG. 5.

### Example 3: Preparation of sodium (3-(4-(5-methyl-1,2,4-oxadiazol-3-yl)thiazole-2-carbonyl)-1H-indol-1-yl)phosphonate

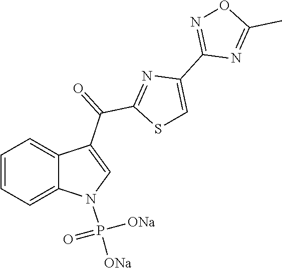

Step 1. Potassium hexamethyldisilazide (0.5 M in toluene) (7.99 ml, 4.00 mmol) was added to an ice-cold suspension of (1H-indol-3-yl)(4-(5-methyl-1,2,4-oxadiazol-3-yl)thiazol-2-yl)methanone (1.24 g, 4.00 mmol) in THF (30 ml). After 20 min, dibenzyl phosphorochloridate (0.3 M in toluene) (13.32 ml, 4.00 mmol) was added to the reaction mixture. The reaction mixture was then allowed to slowly warm to room temperature overnight. Next day, the reaction was treated with saturated NaHCO₃ (aq) and diluted with EtOAc. The organic layer was washed with brine, dried over Na₂SO₄, filtered, and concentrated to give crude dibenzyl (3-(4-(5-methyl-1,2,4-oxadiazol-3-yl)thiazole-2-carbonyl)-1H-indol-1-yl)phosphonate (4.00 mmol). The crude material was taken on as is. ESI MS m/z 577 [M+H]⁺.

Step 2. Bromotrimethylsilane (2.64 ml, 20.00 mmol) was added to an ice-cold suspension of crude dibenzyl (3-(4-(5-methyl-1,2,4-oxadiazol-3-yl)thiazole-2-carbonyl)-1H-indol-1-yl)phosphonate (4.00 mmol) in anhydrous acetonitrile (100 ml). The reaction was allowed to slowly warm to room temperature. Upon completion, the reaction mixture was concentrated under reduced pressure. The residue was treated with CH₂Cl₂ and saturated NaHCO₃ (aq). The CH₂Cl₂ layer was discarded and the aqueous layer was washed twice with additional CH₂Cl₂. Reverse phase chromatography (C18, 5% CH₃CN/H₂O) of the aqueous layer gave sodium (3-(4-(5-methyl-1,2,4-oxadiazol-3-yl)thiazole-2-carbonyl)-1H-indol-1-yl)phosphonate (945 mg, 68%) as a yellow solid after lyophilization.

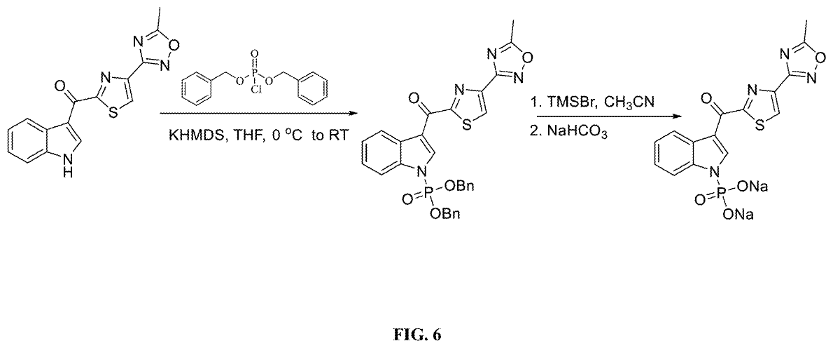

The synthetic approach is shown in FIG. 6.

### Example 4: Preparation of sodium (3-(4-propionylthiazole-2-carbonyl)-1H-indol-1-yl)phosphonate

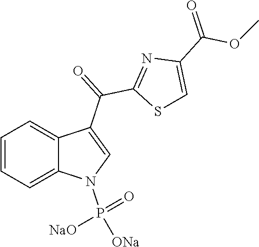

Step 1. Potassium hexamethyldisilazide (0.5 M in toluene) (49.2 ml, 24.62 mmol) was added to an ice-cold mixture of 1-(2-(1H-indole-3-carbonyl)thiazol-4-yl)propan-1-one (7.0 g, 24.6 mmol) in THF (125 ml). After 20 min, dibenzyl phosphorochloridate (0.3 M in toluene) (86 ml, 25.8 mmol) was added and the reaction was allowed to slowly warm to room temperature. After stirring overnight, the mixture was concentrated to dryness. The resulting residue was partitioned between EtOAc and saturated NaHCO₃ (aq). The organic layer was washed with brine, dried over Na₂SO₄, filtered, and then concentrated to yield crude dibenzyl (3-(4-propionylthiazole-2-carbonyl)-1H-indol-1-yl)phosphonate as a yellow oil (24.6 mmol). The crude material was taken on as is. ESI MS m/z 545 [M+H]⁺.

Step 2. Bromotrimethylsilane (10.2 ml, 79.0 mmol) was added to an ice-cold solution of crude dibenzyl (3-(4-propionylthiazole-2-carbonyl)-1H-indol-1-yl)phosphonate (13.4 g, 24.6 mmol) in acetonitrile (100 ml). Next, the ice-bath was removed and the reaction was allowed to warm to room temp. Upon completion, saturated NaHCO₃ (75 ml, 86 mmol) and H₂O were added and the resulting solid was removed by filtration through a Celite pad. The aqueous phase was extracted with CH₂Cl₂ to remove BnBr. The aqueous layer was then purified by reverse phase chromatography (C18, 455 g, 5% CH₃CN/H₂O to CH₃CN) to yield sodium (3-(4-propionylthiazole-2-carbonyl)-1H-indol-1-yl)phosphonate (3.87 g, 38%) as a yellow solid after lyophilization.

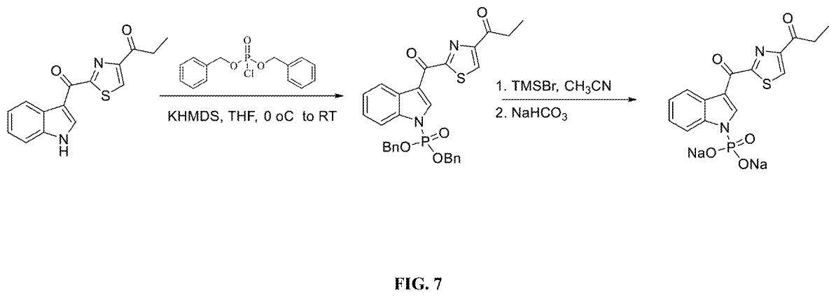

The synthetic approach is shown in FIG. 7.

### Example 5: Preparation of sodium benzyl (5-fluoro-3-(4-propionylthiazole-2-carbonyl)-1H-indol-1-yl)phosphonate

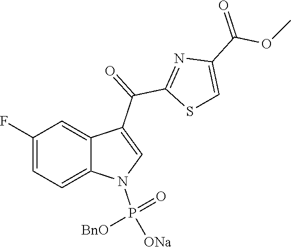

Step 1. Dibenzyl phosphorochloridate (0.112 g/mL in toluene) (24.31 mL, 7.29 mmol) was added to a suspension of 1-(2-(5-fluoro-1H-indole-3-carbonyl)thiazol-4-yl)propan-1-one (2.1 g, 6.95 mmol) in THF (100 mL) and triethylamine (2.90 mL, 20.84 mmol). The mixture was then stirred at room temp. Upon completion, the reaction mixture was concentrated and the residue was treated with EtOAc and saturated NaHCO₃ (aq). The aqueous layer was washed twice with EtOAc. The combined organic phases were then washed with brine, dried over Na₂SO₄, filtered, and concentrated. After determining that the material was the triethylammonium salt, the residue was partitioned between 1 M HCl (aq) and EtOAc. The aqueous layer was extracted a second time with EtOAc. The combined organic phases were washed with saturated NaCl (aq), dried over Na₂SO₄, filtered, and concentrated to yield crude benzyl hydrogen (5-fluoro-3-(4-propionylthiazole-2-carbonyl)-1H-indol-1-yl)phosphonate as a pale yellow solid (2.23 g). ESI MS m/z 577 [M+H]⁺.

Step 2. Sodium bicarbonate (100 mg, 1.190 mmol) was added to a suspension of crude benzyl hydrogen (5-fluoro-3-(4-propionylthiazole-2-carbonyl)-1H-indol-1-yl)phosphonate (237 mg, 0.502 mmol) in water (5 ml). Reverse phase chromatography (C18, 5% CH₃CN/H₂O to CH₃CN) gave sodium benzyl (5-fluoro-3-(4-propionylthiazole-2-carbonyl)-1H-indol-1-yl)phosphonate (193 mg, 77%) as an off-white solid after lyophilization and subsequent drying in a high vacuum oven overnight.

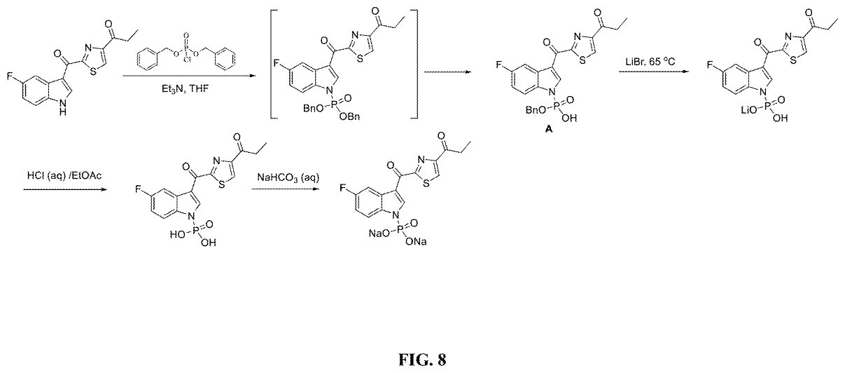

The synthetic approach is shown in FIG. 8.

### Example 6: Preparation of sodium (5-fluoro-3-(4-propionylthiazole-2-carbonyl)-1H-indol-1-yl)phosphonate 1-yl)phosphonate

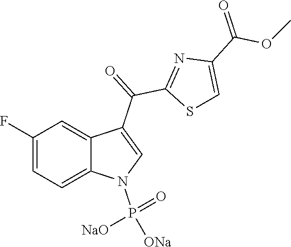

A suspension of crude benzyl hydrogen (5-fluoro-3-(4-propionylthiazole-2-carbonyl)-1H-indol-1-yl)phosphonate (1.78 g, 3.77 mmol) and lithium bromide (1.636 g, 18.84 mmol) in acetonitrile (100 mL) was heated to 65° C. overnight. Next, the reaction mixture was concentrated under reduced pressure and then partitioned between EtOAc and 1N HCl (aq). After separation, the aqueous layer was treated with solid NaCl and then as a precaution, it was extracted four times with EtOAc. The combined organic phases were concentrated to dryness. Next, sodium bicarbonate (0.8 g, 9.52 mmol) and a minimum amount of water were added. The mixture was then extracted with CH₂Cl₂ to remove BnBr. The aqueous layer was briefly placed on the rotovap to remove residual CH₂Cl₂ and then filtered. Reverse phase chromatography (C18, 5% CH₃CN/H₂O) of the filtrate gave sodium (5-fluoro-3-(4-propionylthiazole-2-carbonyl)-1H-indol-1-yl)phosphonate (1.12 g, 69%) as a yellow solid after lyophilization.

The synthetic approach is shown in FIG. 8. Stepwise phosphonate deprotection of the benzyl group with Et₃NHCl in situ to give intermediate A (after isolation) minimizes unwanted nitrogen phosphorous bond cleavage observed with the TMSBr/CH₃CN deprotection of the phosphonate diester. Intermediate A (or salt thereof) has improved stability over the phosphonate diester toward hydrolitic cleavage.

### Example 7: Preparation of sodium (3-(4-(5-amino-1,3,4-oxadiazol-2-yl)thiazole-2-carbonyl)-1H-indol-1-yl)phosphonate

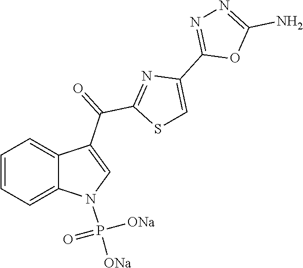

Step 1. Potassium hexamethyldisilazide (0.5 M in toluene) (6.42 ml, 3.21 mmol) was added to a suspension of (4-(5-amino-1,3,4-oxadiazol-2-yl)thiazol-2-yl)(1H-indol-3-yl)methanone (1.0 g, 3.21 mmol) in pyridine (40 ml) at room temperature. After 20 min, the solution was cooled in an ice bath and di-tert-butyl phosphorochloridate (17.52 ml, 3.85 mmol) was added. After stirring overnight, the mixture was concentrated. The residue was treated with EtOAc and 1 N HCl (aq). The organic layer was concentrated to yield primarily tert-butyl hydrogen (3-(4-(5-amino-1,3,4-oxadiazol-2-yl)thiazole-2-carbonyl)-1H-indol-1-yl)phosphonate (3.21 mmol), as judged by its retention time on the LCMS. By mass, it was identified as the des-tert-butyl product ESI MS m/z 392 [M+H]⁺. The crude material was taken on as is.

Step 2. Crude tert-butyl hydrogen (3-(4-(5-amino-1,3,4-oxadiazol-2-yl)thiazole-2-carbonyl)-1H-indol-1-yl)phosphonate (3.21 mmol) was treated with acetone (16 mL) and water (16 ml), and the resulting solution was heated to 50° C. overnight. Following day, saturated NaHCO₃ was added to the reaction mixture until it was basic. The mixture was then concentrated to dryness to remove acetone. Next, water was added and the mixture was filtered through Celite to remove solids. Reverse phase chromatography (C18, 5% CH₃CN/H₂O) of the filtrate gave sodium (3-(4-(5-amino-1,3,4-oxadiazol-2-yl)thiazole-2-carbonyl)-1H-indol-1-yl)phosphonate (435 mg, 31%) as a yellow solid after lyophilization.

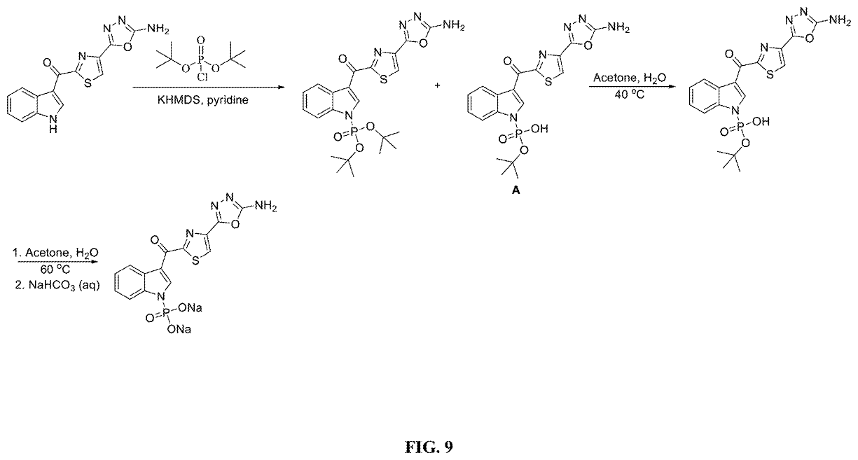

The synthetic approach is shown in FIG. 9. Heating in acetone/water gave controlled, stepwise deprotection of the t-butyl protecting groups. Deprotection was achieved by mildly heating intermediate A, as the free acid or the sodium salt, with AcOH or TFA. Intermediate A was isolated as the sodium salt. Intermediate A (or salt thereof) has improved stability over the phosphonate diester.

Collectively, Examples 5-7 demonstrate stepwise, controlled deprotection of either the benzyl or t-butyl phosphorous protecting groups to yield phosphonate diesters or intermediates of type A (e.g. t-butyl or benzyl) that can be further deprotected to yield the final N-phosphonate compound.

### Example 8: In Vivo Pharmacokinetic Studies in Dogs and Rats

This example describes pharmacokinetic (PK) studies of ARI-158 and ARI-160 in dogs and rats, respectively. In the present studies, the test compounds were given to groups of male beagle dogs and rats (N=3 in each group) intravenously (IV) at 2 mg/kg or orally (PO) at 10 mg/kg. IV doses were formulated in DMSO, while PO doses were formulated in a 50/50 mixture of PEG400 and Tween 80. Blood samples were collected at pre-dose and over a period of 24 hours post-dose. Plasma concentrations of the indolo-phosphoramidate compounds were determined by HPLC. Tables 2a-c below show the results of the PK studies.

TABLE 2a
PK Studies in Dogs Following a 10 mg/kg Oral Dose of ARI-158

| | Time | Plasma Concentration (ng/mL) | | | | |
|---|---|---|---|---|---|---|
| Analyte | (h) | Dog 10 | Dog 11 | Dog 12 | Mean | SD |
| ARI-158 | 0 | BLQ | BLQ | BLQ | N/A | BLQ |
| | 0.083 | 153 | 149 | 14.3 | 105 | 78.9 |
| | 0.25 | 438 | 414 | 87.3 | 313 | 196 |
| | 0.5 | 786 | 561 | 178 | 508 | 307 |
| | 1 | 677 | 311 | 275 | 421 | 222 |
| | 2 | 128 | 102 | 158 | 129 | 28.0 |
| | 4 | 11.2 | 14.4 | 69.8 | 31.8 | 32.9 |
| | 6 | 3.28 | 1.47 | 14.1 | 6.28 | 6.83 |
| | 8 | 1.47 | BLQ | 4.62 | 3.05 | 2.23 |
| | 24 | BLQ | BLQ | BLQ | N/A | BLQ |
| ARI-143 | 0 | 1.91 | BLQ | BLQ | 1.91 | BLQ |
| | 0.083 | 1.28 | 67.2 | 107 | 58.5 | 53.4 |
| | 0.25 | 610 | 1080 | 1120 | 937 | 284 |
| | 0.5 | 1950 | 1710 | 1260 | 1640 | 350 |
| | 1 | 1540 | 971 | 1080 | 1200 | 302 |
| | 2 | 310 | 269 | 284 | 288 | 20.7 |
| | 4 | 24.1 | 19.1 | 14.6 | 19.3 | 4.75 |
| | 6 | 1.51 | BLQ | BLQ | 1.51 | BLQ |
| | 8 | BLQ | BLQ | BLQ | N/A | BLQ |
| | 24 | 1.07 | BLQ | BLQ | 1.07 | BLQ |

N/A = Not Applicable
BLQ = Below the Limit of Quantitation

TABLE 2b
PK Parameters of ARI-158 and ARI-143 in Dogs
Given a 10 mg/kg Oral Dose of ARI-158

| | | Tₘₐₓ | Cₘₐₓ | AUCₗₐₛₜ | AUC_{INF} | t_{1/2} |
|---|---|---|---|---|---|---|
| Analyte | Subject | (h) | (ng/mL) | (h*ng/mL) | (h*ng/mL) | (h) |
| ARI-158 | Dog 10 | 0.500 | 786 | 1020 | 1020 | 1.37 |
| | Dog 11 | 0.500 | 561 | 675 | 677 | 0.654 |
| | Dog 12 | 1.000 | 275 | 669 | 676 | 1.02 |
| | Mean | 0.667 | 541 | 787 | 791 | 1.01 |
| | SD | 0.289 | 256 | 199 | 198 | 0.356 |
| ARI-143 | Dog 10 | 0.500 | 1950 | 2270 | | |
| | Dog 11 | 0.500 | 1710 | 1840 | | |
| | Dog 12 | 0.500 | 1260 | 1770 | | |
| | Mean | 0.500 | 1640 | 1960 | | |
| | SD | 0.000 | 350 | 273 | | |

TABLE 2c
PK Studies in Rats Following a 2 mg/kg IV Dose or 10 mg/kg Oral Dose of ARI-160
ARI-160 PK Parameters

| | | Dosage | | Tmax | Cmax | T½ | MRTlast | MRTinf | AUClast | AUCinf | Cl | Vss |
|---|---|---|---|---|---|---|---|---|---|---|---|---|
| Dosed Compound | Route | (mg/kg) | Animal_ID | (hr) | (ng/mL) | (hr) | (hr) | (hr) | (hr*ng/mL) | (hr*ng/mL) | (mL/hr/kg) | (mL/kg) |
| ARI-160 | IV | 2 | 1 | 0.083 | 16100 | 0.62 | 0.21 | 0.23 | 5120 | 5140 | 389 | 89.8 |
| | | | 2 | 0.083 | 12100 | 0.86 | 0.23 | 0.26 | 3930 | 3950 | 506 | 132 |
| | | | 3 | 0.083 | 24600 | 0.90 | 0.18 | 0.18 | 8590 | 8590 | 233 | 42 |
| | | | Mean | 0.083 | 17600 | 0.79 | 0.21 | 0.22 | 5880 | 5890 | 376 | 87.8 |
| | | | SD | 0 | 6380 | 0.15 | 0.03 | 0.04 | 2420 | 2410 | 137 | 44.8 |
| | | | CV % | 0 | 36.3 | 19.1 | 12.8 | 18 | 41.2 | 40.9 | 36.4 | 51 |

| | | Dosage | | Tmax | Cmax | T½ | MRTlast | MRTinf | AUClast | AUCinf | F. |
|---|---|---|---|---|---|---|---|---|---|---|---|
| Dosed Compound | Route | (mg/kg) | Animal_ID | (hr) | (ng/mL) | (hr) | (hr) | (hr) | (hr*ng/mL) | (hr*ng/mL) | (%) |
| ARI-160 | PO | 10 | 4 | 0.25 | 12.7 | 1.58 | 0.864 | 2.32 | 13.3 | 23.3 | |
| | | | 5 | 0.25 | 10.1 | 6.14 | 3.25 | 8.41 | 32.1 | 51.8 | |
| | | | 6 | 0.25 | 6.75 | 6.07 | 2.01 | 9.57 | 11.0 | 22.5 | |
| | | | Mean | 0.25 | 9.85 | 4.59 | 2.04 | 6.77 | 18.8 | 32.5 | 0.110 |
| | | | SD | 0 | 2.98 | 2.61 | 1.19 | 3.90 | 11.6 | 16.7 | |
| | | | CV % | 0 | 30.3 | 56.9 | 58.4 | 57.6 | 61.6 | 51.3 | |

The PK data for ARI-158 show that for oral administration in dogs, ARI-158 (the “prodrug”) delivered ARI-143 (the “drug”) to the plasma much more effectively than an equivalent dose of ARI-143 itself. Also, ARI-158 was absorbed much more efficiently than ARI-143. In the case of ARI-160, the PK data show that ARI-160 exhibited better bioavailability with an IV dosing. Thus, both ARI-158 and ARI-160 provided ARI-143 after oral as well as IV administration.

### Example 9: Anti-Tumor Activity of ARI-158 and ARI-160 in Animal Models

This example describes in vivo studies that evaluated the anti-cancer efficacy of ARI-158 and ARI-160 in syngeneic mouse tumor models. Mice implanted subcutaneously with EMT-6 cancer cells were treated with ARI-158 and ARI-160, or vehicle controls, as described below.

Materials and Methods

Cell Culture

A monolayer culture of tumor cells was maintained in vitro in DMEM or RPMI1640 medium supplemented with 10% fetal bovine serum at 37° C. in an atmosphere of 5% CO₂. Cells in exponential growth phase were harvested and quantitated by cell counter before tumor inoculation.

Subcutaneous Syngeneic Mouse Tumor Models

EMT-6 syngeneic mouse tumor models were generated by inoculating female BALB/C 6 mice with EMT-6 cancer cells at their right lower flank.

Each mouse was inoculated subcutaneously with tumor cells in 0.1 mL of PBS. Treatments were started when the mean tumor size reached approximately 80-120 mm³ (around 100 mm³). The administration of the test compounds and the animal number in each study group are shown in the study design. The date of tumor cell inoculation was denoted as day 0.

Formulation of Test Compounds

ARI-158 and ARI-160 were dissolved in DMSO at the final concentration of 26.7 mg/ml and stored at room temperature.

Study Design

Randomization of animals was started when the mean tumor size reached approximately 90 mm³ to form the mouse study groups. The randomization was performed based on “Matched distribution” method using the multi-task method (StudyDirector™ software, version 3.1.399.19)/randomized block design. The mouse groups (ten in each group) were treated with vehicle (DMSO) or the test compounds at a dose of 40 mg/kg by i.p. injection, QD for 28 days or longer.

Observation and Data Collection

After tumor cell inoculation, the mice were checked daily for morbidity and mortality. During routine monitoring, the mice were checked for tumor growth and any effects of the treatment on behavior such as mobility, food and water consumption, body weight gain/loss (body weights were measured twice per week after randomization), eye/hair matting, and any other abnormalities. Mortality and observed clinical signs were recorded for individual mice in detail.

Tumor volumes were measured twice per week in two dimensions using a caliper, and the volume was expressed in mm³ using the formula:

V=(L×W×W)/2,

where V is tumor volume, L is tumor length (the longest tumor dimension) and W is tumor width (the longest tumor dimension perpendicular to L). Dosing as well as tumor and body weight measurements was conducted in a Laminar Flow Cabinet. The body weights and tumor volumes were measured by using StudyDirector™ software (version 3.1.399.19).

Dosing Holiday

A dosing holiday was given to the mice after one measurement of body weight loss (BWL)>30%. The length of the dosing holiday was long enough for the body weight to recover to BWL<30%, at which time the treatment was resumed. The mice were not fed any additional nutrient supplement during the dosing holiday.

### Experimental Termination

Tumor growth inhibition percentage (TGI %) is an indicator for antitumor activity of a drug compound, and expressed as:

TGI (%)=100×(1−T/C),

where T and C are the mean tumor volume (or weight) of the treated and control groups, respectively, on a given day. Statistical analysis of the difference in mean tumor volume (MTV) among the groups was conducted using the data collected on the day when the MTV of the vehicle group reached the humane endpoints, so that TGI could be derived for all or most mice enrolled in the study.

The body weight of all animals was monitored throughout the study and animals were euthanized if they lost over 20% of their body weight relative to the weight at the start of the study and could not recover within 72 hours.

All of the mice in the same group would be sacrificed when the MTV reached 2000 mm³, or an individual mouse would be sacrificed when the tumor volume reached 3000 mm³.

To deter cannibalization, any animal exhibiting an ulcerated or necrotic tumor would be separated immediately and singly housed and monitored daily before the animal was euthanized or until tumor regression was complete. Mouse with tumor ulceration of approximately 25% or greater on the surface of the tumor would be euthanized.

Statistical Analysis

For comparison between two groups, a Student's t-test was performed. All data were analyzed using SPSS 18.0 and/or GraphPad Prism 5.0. P<0.05 was considered statistically significant.

Results

In vivo studies were performed in the above-described syngeneic mouse tumor model to evaluate the anti-tumor activity of ARI-158 and ARI-160. This syngeneic mouse tumor model was chosen for this study because EMT-6 provides a fast-growing model that enables relatively quick differentiation of performance between compounds. Table 3 summarizes the tumor growth inhibition (TGI) data collected on the indicated days post tumor inoculation. The vehicle arm was terminated on the indicated days (D), when the vehicle control mice reached the humane endpoints.

TABLE 3
TGI data - ARI-158 vs ARI-160
Study Days

| Compound | 6 | 10 | 13 | 17 | 20 | 24 | 27 | 31 |
|---|---|---|---|---|---|---|---|---|
| ARI-158 (80 mpk) | −0.01% | 2.92% | −1.75% | 17.21% | 38.27% | 44.41% | 42.16% | 39.97% |
| ARI-160 (80 mpk) | 0.01% | 30.95% | 30.58% | 20.47% | 32.25% | 33.19% | 22.24% | 12.40% |

% T/C = mean(T)/mean(C) * 100
T—current group value
C—control group value

The TGI data show that ARI-158 at 80 mpk had better efficacy than ARI-160.

What is claimed is:

1. A compound of Formula I: 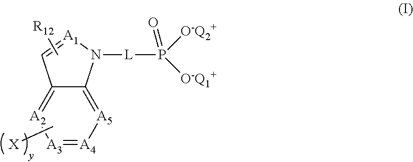
R₁₂ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio,
each of A₁, A₂, A₃, A₄, and A₅, independently, is CR₂ or N;
L is —(CR₂R₃—O)ₙ— or a bond;
R₂ is H or C₁-C₆ alkyl;
R₃ is H or C₁-C₆ alkyl;
or, together, R₂ and R₃ form a C₃-C₈ cycloalkyl;
n is 0, 1, 2, 3, 4, 5, or 6;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio; and
Q₁⁺ and Q₂⁺ are each, independently, a monocation, or together are a dication or one or both of Q₁⁺ or Q₂⁺ is H, C₁-C₆ alkyl, allyl, benzyl, or —(CR₂R₃—O)—R₂₃, and R₂₃ is H or C₁-C₆ alkyl.

2. A compound of claim 1, the compound is of Formula II: 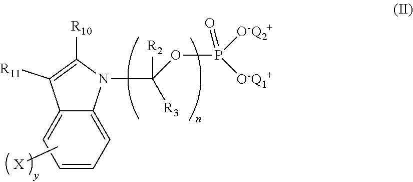
R₁₀ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
R₁₁ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ is H or C₁-C₆ alkyl;
R₂ is H or C₁-C₆ alkyl;
R₃ is H or C₁-C₆ alkyl;
or, together R₂ and R₃ form a C₃-C₈ cycloalkyl;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
Q₁⁺ and Q₂⁺ are each, independently, a monocation, or together are a dication or one or both of Q₁⁺ or Q₂⁺ is H, C₁-C₆ alkyl, allyl, benzyl, or —(CR₂R₃—O)—R₂₃, and R₂₃ is H or C₁-C₆ alkyl; and
n is 0, 1, 2, 3, 4, 5, or 6.

3. A compound of claim 1, wherein the compound is of Formula III: 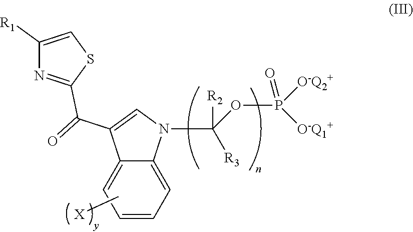
wherein:
R₁ is —C(═O)—R₄, cyano, an oxadiazole, or a thiadiazole;
R₂ and R₃ are each, independently, hydrogen, or C₁-C₆ alkyl; and
R₄ is selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
Q₁⁺ and Q₂⁺ is each, independently, a monocation, or together are a dication or one or both of Q₁⁺ or Q₂⁺ is H, C₁-C₆ alkyl, benzyl, allyl or —(CR₂R₃—O)—R₂₃, and R₂₃ is H or C₁-C₆ alkyl, and the other of Q₁⁺ or Q₂⁺ can be a monocation; and
n is 0, 1, 2, 3, 4, 5, or 6.

4. A compound of claim 1, wherein the compound is of Formula IV: 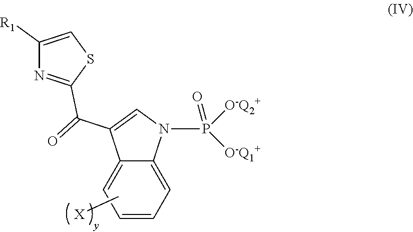
wherein:
R₁ is —C(═O)—R₄, cyano, an oxadiazole, or a thiadiazole; and
R₄ is selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio; and
Q₁⁺ and Q₂⁺ is each, independently, a monocation, or together are a dication or one or both of Q₁⁺ or Q₂⁺ is H, C₁-C₆ alkyl, allyl, benzyl, or —(CR₂R₃—O)—R₂₃, and R₂₃ is H or C₁-C₆ alkyl, and the other of Q₁⁺ or Q₂⁺ can be a monocation.

5. A compound of claim 1, wherein the compound is of Formula V: 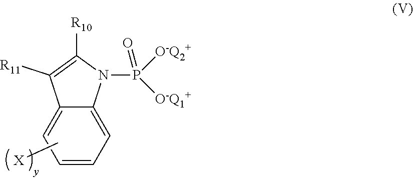
wherein:
R₁ is —C(═O)—R₄, cyano, an oxadiazole, or a thiadiazole;
R₂ and R₃ are each independently hydrogen, or C₁-C₆ alkyl; and
R₄ is selected from the group consisting of —NRₐR_{b} (Rₐ and R_{b} are each independently H, C₁-C₆ alkyl, or C₁-C₆ acyl), hydrogen, deuterium, halo, amino, hydroxy, cyano, formyl, furyl, nitro, alkyl, haloalkyl, alkenyl, haloalkenyl, alkynyl, haloalkynyl, unsubstituted or substituted C₁-C₆ acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, halothiocarbonylthio, and S(O)ₘR₂₂ (m=0 to 2, R₂₂ is directly connected to S), wherein R₂₂ is selected from the group consisting of hydrogen, deuterium, halo, amino, hydroxy, thiol, cyano, formyl, alkyl, haloalkyl, alkenyl, haloalkenyl, alkynyl, haloalkynyl, acyloxy, alkoxy, haloalkoxy, thioalkoxy, halothioalkoxy, alkanoyl, haloalkanoyl, thioalkanoyl, halothioalkanoyl, carbonyloxy, halocarbonyloxy, carbonylthio, halocarbonylthio, thiocarbonyloxy, halothiocarbonyloxy, thiocarbonylthio, and halothiocarbonylthio;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio; and
Q₁⁺ and Q₂⁺ is each, independently, a monocation, or together are a dication or one or both of Q₁⁺ or Q₂⁺ is H, C₁-C₆ alkyl, allyl, benzyl, or —(CR₂R₃—O)—R₂₃, and R₂₃ is H or C₁-C₆ alkyl, and the other of Q₁⁺ or Q₂⁺ can be a monocation.

6. The compound of any one of claims 1-5, wherein Q₁⁺ and Q₂⁺ are each, independently, an alkali metal.

7. The compound of any one of claims 1-5, wherein Q₁⁺ and Q₂⁺ are each, independently, selected from the group consisting of ammonium and alkyl ammonium.

8. The compound of any one of claims 1-5, wherein Q₁⁺ and Q₂⁺ together are an alkaline earth metal salt.

9. The compound of any one of claims 1-5, wherein Q₁⁺ and Q₂⁺ are each independently selected from the group consisting of zinc, calcium, and magnesium.

10. The compound of any one of claims 1-5, wherein Q₁⁺ and Q₂⁺ are each independently lithium, sodium, or potassium, y is 0, 1, or 2, and X is F, Cl, or Br.

11. The compound of any one of claim 3 or 4, wherein R₁ is —C(═O)—R₄, and R₄ is C₁-C₆ alkyl or C₁-C₆ alkoxy.

12. The compound of any one of claim 3 or 4, wherein R₁ is an oxadiazole or a thiadiazole, wherein the oxadiazole, or the thiadiazole is optionally substituted by amino, alkyl amino, amino alkyl, alkoxy, alkyl, or haloalkyl.

13. The compound of any one of claims 1-3, wherein n is 0 or 1.

14. The compound of claim 2, wherein the compound is selected from the group consisting of:

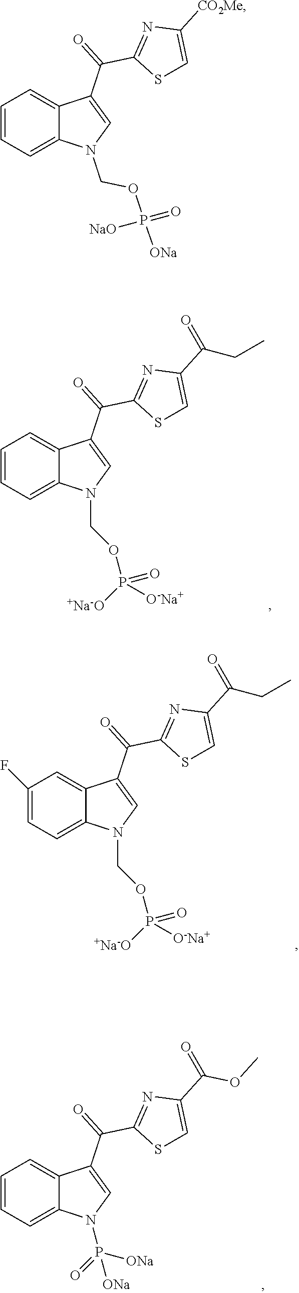

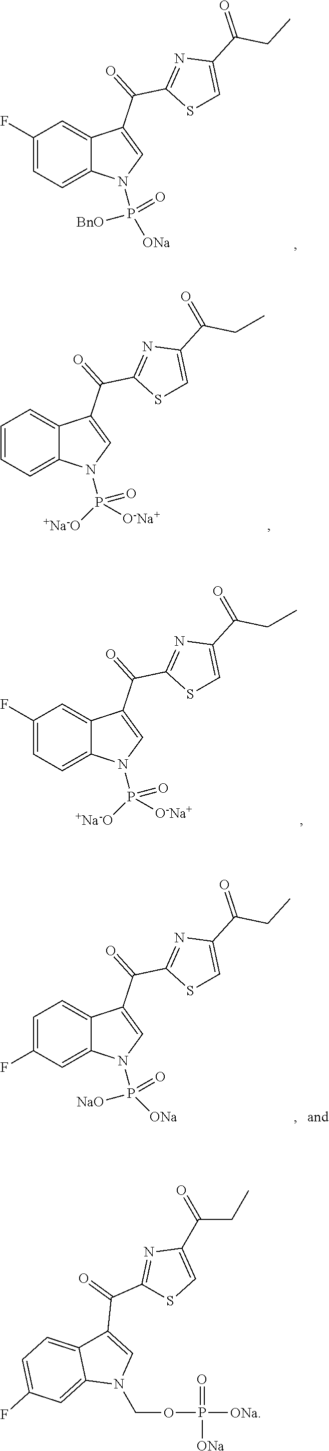

15. The compound of any one of claim 3 or 4, wherein R₁ is an unsubstituted or substituted oxadiazole.

16. The compound of claim 15 wherein the substituted oxadiazole is a C₁-C₆ alkyl oxadiazole, haloalkyl oxadiazole, halo oxadiazole, amino oxadiazole, alkyl amino oxadiazole, amino alkyl oxadiazole, or hydroxy oxadiazole.

17. The compound of claim 16, wherein n is 0.

18. The compound of claim 17, wherein Q₁⁺ and Q₂⁺ are each lithium, sodium, or potassium.

19. The compound of claim 16, wherein the indole is a fluorinated indole.

20. The compound of claim 2, wherein the compound is selected from the group consisting of:

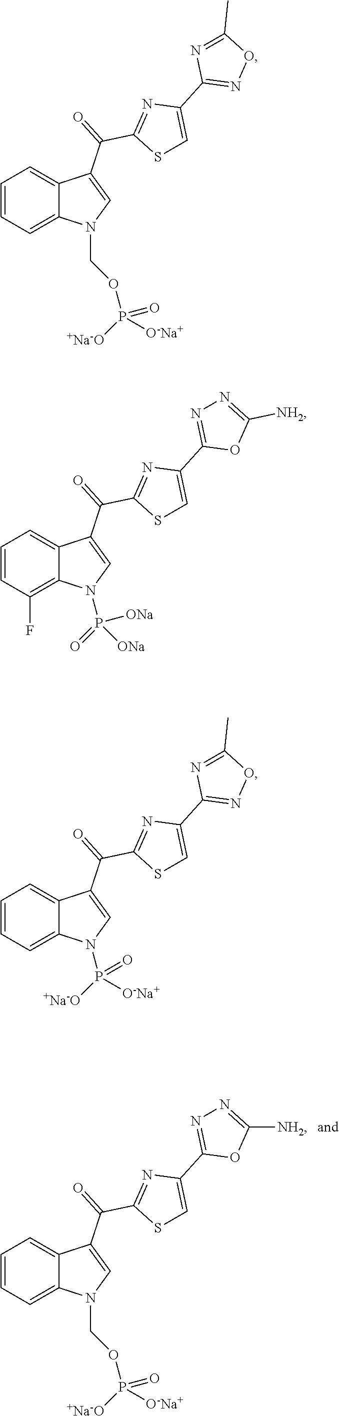

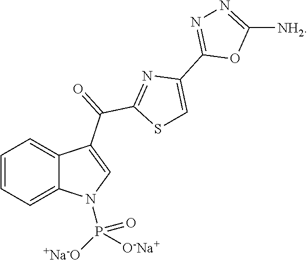

21. A compound of Formula VI: 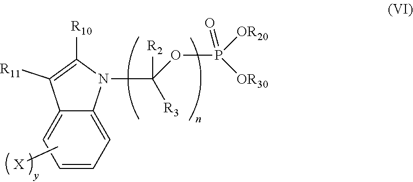
R₁₀ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
R₁₁ is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, alkanoyl, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio, wherein one of R₁₀ and R₁₁ is H or C₁-C₆ alkyl;
R₂ is H or C₁-C₆ alkyl;
R₃ is H or C₁-C₆ alkyl;
or, together, R₂ and R₃ form a C₃-C₈ cycloalkyl;
y is 0, 1, 2, 3, or 4;
each X, independently, is hydrogen, deuterium, halo, amino, hydroxy, thiol, cyano, formyl, alkyl, haloalkyl, alkenyl, haloalkenyl, alkynyl, haloalkynyl, acyl, acyloxy, alkoxy, haloalkoxy, thioalkoxy, halothioalkoxy, haloalkanoyl, thioalkanoyl, halothioalkanoyl, carboxy, carbonyloxy, halocarbonyloxy, carbonylthio, halocarbonylthio, thiocarbonyloxy, halothiocarbonyloxy, thiocarbonylthio, or halothiocarbonylthio;
R₂₀ and R₃₀ each, independently, is H, C₁-C₆ alkyl, allyl, or benzyl, or one of R₂₀ or R₃₀ is H, C₁-C₆ alkyl, allyl, or benzyl and the other of R₂₀ or R₃₀ is a cation; and
n is 0, 1, 2, 3, 4, 5, or 6.

22. A method of enhancing the immune system in a patient in need thereof, comprising administering to the patient a therapeutically effective amount of a compound of any one of claims 1-21.

23. Use of a compound of any one of claims 1-21 for the manufacture of a medicament for enhancing the immune system in a patient in need thereof.

24. A compound of any one of claims 1-21 for use in enhancing the immune system in a patient in need thereof.

25. The method of claim 22, use of claim 23, or compound for use of claim 24, wherein the patient has cancer, is immune compromised, or has an infection.

26. A method of treating cancer in a patient in need thereof, comprising administering to the patient a therapeutically effective amount of the compound of any one of claims 1-21.

27. Use of a compound of any one of claims 1-21 for the manufacture of a medicament for treating cancer in a patient in need thereof.

28. A compound of any one of claims 1-21 for use in treating cancer in a patient in need thereof.

29. The method of claim 26, use of claim 27, or compound for use of claim 28, wherein the cancer is a hematological malignancy or a solid tumor.

30. The method of claim 26, use of claim 27, or compound for use of claim 28, wherein the cancer is selected from the group consisting of diffuse large B-cell lymphoma, marginal zone lymphoma, chronic lymphocytic leukemia, small lymphocytic lymphoma, prolymphocytic leukemia, acute lymphocytic leukemia, Waldenstrom's Macroglobulinemia, follicular lymphoma, mantle cell lymphoma, Hodgkin lymphoma, non-Hodgkin lymphoma, multiple myeloma, prostate cancer, ovarian cancer, fallopian tube cancer, cervical cancer, breast cancer, lung cancer, skin cancer, colorectal cancer, stomach cancer, pancreatic cancer, liver cancer, kidney cancer, bladder cancer, soft tissue cancer, glioma, and head and neck cancer.
